(19) United States
(12) Patent Application Publication
(10) Pub. No.: US 2005/0245466 A1
(43) Pub. Date: Nov. 3, 2005

(54) Use of topoisomerase inhibitors and heat shock protein 90 inhibitors for use in chemotherapy

(75) Inventor: John Jenkins, Liverpool (GB)

Correspondence Address: PILLSBURY WINTHROP SHAW PITTMAN LLP, 725 S. FIGUEROA STREET, SUITE 2800, LOS ANGELES, CA, 90017, US

(73) Assignee: THE UNIVERSITY OF LIVERPOOL, Liverpool (GB)

(21) Appl. No.: 10/509,143

(22) PCT Filed: Mar. 28, 2003
(86) PCT No.: PCT/GB03/01369

§ 371 Date: Jul. 15, 2005

(30) Foreign Application Priority Data
Mar. 28, 2002 (GB) 0207362.5

(51) Int. Cl. A61K 48/00; A61K 31/7072; A61K 31/7048; A61K 31/704; A61K 31/4745; A61K 31/513; A61K 31/407

(52) U.S. Cl. 514/27; 514/34; 514/49; 514/284; 514/270; 514/410; 514/44; 514/255.02

(57) ABSTRACT

The present invention relates to the use of a first agent that attenuates topoisomerase II (Topo II) activity and a second agent that inhibits Heat Shock Protein 90 (HSP90) for use in chemotherapy. The agents are particularly useful in the treatment of cancer and destruction of micro-organisms. The invention also relates to screening methods, diagnostic methods and methods for evaluating or monitoring chemotherapy regimens.

The present invention relates to the treatment of medical conditions using a combination of chemotherapeutic agents.

In general, when chemotherapy is used for the treatment of human cancers and the like, a combination of agents is employed. In the past, the reasoning behind the choice of which particular combinations of agents are used has been essentially a pragmatic decision, often based more on tolerances to toxicity rather than specific targets.

Recent studies of the process of carcinogenesis, have revealed that many of the genetic lesions involved, cause errors in the cell division/death pathways. The molecular changes that result from such lesions initiate the cancer process. Due to this the molecules involved in such changes provide potentially highly specific targets for chemotherapy. Using the targets identified by this approach new therapeutic agents may be introduced into the clinic. However, to achieve optimal clinical benefit from these agents, they may too need to be used in combination with other anticancer drugs. Again the choice of which particular combinations of agents are used has been a decision based more on tolerances to toxicity rather than specific targets.

There is also a need to develop new and improved antimicrobial agents. Antibiotic resistance is a growing problem and there is an increasing need to provide effective combination therapies.

According to a first aspect of the present invention, there is provided a use of a first agent that attenuates Topoisomerase II activity and a second agent that inhibits Heat Shock Protein 90 activity for the manufacture of a medicament for contemporaneous or sequential administration in chemotherapy.

According to a second aspect of the present invention, there is provided a method for conducting chemotherapy comprising contemporaneously or sequentially administering to a person or animal in need of said treatment a therapeutically effective amount of a first agent that attenuates Topoisomerase II activity and a second agent that inhibits Heat Shock Protein 90 activity.

According to a third aspect of the present invention, there is provided a composition for use in chemotherapy comprising therapeutically effective amounts of a first agent that attenuates Topoisomerase II activity and a second agent that inhibits Heat Shock Protein 90 activity and a pharmaceutically acceptable vehicle.

By “chemotherapy” we mean treatment of cells to cause a targeted cell death. Chemotherapy is required in cancer treatment where it is desirable to target transformed cells. Chemotherapy is also employed to treat infections caused by pathogens (e.g. bacterial, fungal or viral infections).

Topoisomerase II (Topo II) is required for the viability of all eukaryotic cells and plays important roles in DNA replication, recombination, chromosome segregation and the maintenance of the nuclear scaffold. Topoisomerase II is also required for RNA polymerase II transcription from chromatin templates. In human and other mammalian cells, there are at least two forms of the Topoisomerase II enzyme, but in yeast there is only one. The two human isoforms of Topoisomerase II designated α and β show distinct patterns of expression during the cell cycle, having different nuclear localisation and tissue specific expression. However there are no functional differences between the two enzymes in their ability to support chromosomal segregation. Proteins associated with these enzymes modulate their activity and are likely to be isozyme specific. The activity of the yeast topoisomerase II is regulated by its phosphorylation state. One of the most important kinases regulating the yeast Topoisomerase II is casein kinase II (CKII). The interaction between these two proteins is sufficiently strong that CKII co-purifies with topoisomerase II.

Sequences for Topoisomerase II are known to the art and may be found in the following papers/gene databases:
(a) Human Topo II α: Tsai-Pflugfelder et al. Proc Natl Acad Sci USA October 1988; 85(19): 7177-81; GENBANK/J04088; and NCBI PubMed nucleotide LOCUS NM_{—}001067.
(b) Human Topo II β: Jenkins et al. Nucleic Acids Res 1992 Nov. 11;20(21):5587-92 GENBANK/X68060; and NCBI PubMed nucleotide LOCUS NM_{—}001068.
(c) Splice variants exist for Topo II β and are referred to by Davies, S. L. et al. Nucleic Acids Res. 21 (16), 3719-3723 (1993); GENBANK/X71911; and NCBI PubMed nucleotide LOCUS HSTOPIIB2
(d) Yeast Topo II: Giaever G et al. J Biol Chem 1986 Sep. 25;261(27):12448-54; GENBANK/M13814; and NCBI PubMed nucleotide LOCUS YSCTOP2
(e) A bacterial homologue of Topo II (DNA gyrase—which has two subunits): Swanberg & Wang J Mol Biol 1987 Oct. 20;197(4):729-36; Adachi et al. Nucleic Acids Res 1987 Jan. 26;15(2):771-84; GENBANK/X04341; GENBANK/X00870; and NCBI PubMed nucleotide LOCUS ECGYRBF

Some known chemotherapeutic agents are believed to act as Topo II inhibitors. They are mainly used in the treatment of acute cancer, particularly leukaemia for remission induction, as salvage therapy and conditioning therapy for bone marrow transplantation. One of the main drugs used is etoposide (VP16).

However, agents such as etoposide have problems with toxicity in healthy tissues. Furthermore resistance often develops and complicates the treatment of cancers (e.g. leukaemias). Such drugs can also induce sister chromatid exchange, chromosomal recombination and chromosome aberrations and are associated with a significant risk of secondary leukaemia. Various factors that may modulate cell death and apoptosis in response to topoisomerase II inhibition include the p53 status of the cell, levels and activity of the Bax and Bcl-2 families.

Heat Shock Protein 90 (HSP90) consists of a highly conserved, 25 kDa N-terminal domain connected to a highly conserved, 55 kDa C-terminal region by a ‘charged linker’, which is variable in both length and composition among species and isoforms. The eukaryotic HSP90s are essential and ubiquitous molecular chaperones with key roles in the folding, activation and assembly of a range of client proteins typically involved in signal transduction, cell cycle control or transcriptional regulation.

Sequences for HSP90 are known to the art and may be found in the following papers/gene databases:
(a) Human HSP90 beta: Rebbe et al. Gene 1987;53(2-3):235-45; GENBANK/M16660; and NCBI PubMed nucleotide LOCUS HUMHSP90
(B) A bacterial homologue of HSP90 from E.coli (HtpG): Nemoto et al. Eur J Biochem. October 2001;268(20):5258-69; swissprot: locus HTPG_ECOLI, accession P10413 (protein accession number); and NCBI PubMed protein LOCUS HTPG_ECOLI

Heat Shock proteins exert their effect under conditions of stress such as heat shock, oxidative, chemical and other stress situations. The biochemical function of HSP90 is catalysing the correct folding and maturation of a number of protein substrates. Without the function of HSP90 the abnormal conformation of the partner proteins would target them for proteolytic degradation.

HSP90 is known to bind to mediators of signalling pathways and other proteins but it is not known to the art that HSP90 may interact with Topo II. However the inventors have established that HSP90 and Topo II interact.

The inventor has found that the combined use of a first agent that attenuates Topoisomerase II activity and a second agent that inhibits Heat Shock Protein 90 activity is highly effective for effecting chemotherapy. The first and second agents may be administered contemporaneously (e.g. as a composition according to the third aspect of the invention) or sequentially. If administered sequentially the first and second agents should be therapeutically active within the subject being treated at the same time.

Chemotherapy with first and second agents according to the invention is particularly useful because such therapy results in synergistic actions. Furthermore satisfactory therapy may be effected using lower doses than would be required in a monotherapy. This has the advantage that the toxic side-effects associated with high doses of chemotherapeutic agents may be obviated or reduced. For instance, damage to healthy tissues (and other associated side effects of high dose chemotherapy—e.g. sickness, hair loss) may be reduced in human cancer chemotherapy by using lower doses of the combined agents according to the invention (than would be required in a monotherapy) without compromising the efficacy of the treatment.

The invention is based upon our studies that have been orientated towards the rational design of chemotherapeutic regimens. The inventor realised that drug development up to the present time has only been directed against single molecule targets and that rational selection of combination chemotherapy may be based on investigating the mechanisms of action of chemotherapeutic agents and identifying potential interaction at the cellular targets of such agents. The inventor's studies established that Topo II and HSP90 interact and lead to the realisation that a combination of agents that specifically inhibit the individual proteins will have great efficacy in chemotherapy. Further experimentation (see the Example) established that treatment of cells with a combination of agents according to the invention was highly effective as a chemotherapy. Furthermore the combination surprisingly represented a synergistic effect. Although we do not wish to be bound by any hypothesis, we believe that disrupting the interaction between the Topo II and HSP90 allows the generation of more DNA damage, thus killing the dividing cells, than would be possible using the agents in monotherapy. We believe the agents have such efficacy because two targets in a single pathway (the stress response pathway) are modulated.

Two papers in the prior art contemplate the use of HSP90 inhibitors in combination with other chemotherapeutic agents.

Munster et al. (Clin Cancer Res August 2001;7(8):2228-36) discloses that ansamycin antibiotics such as 17-AAG (an HSP90 inhibitor) and Doxorubicin may be combined in chemotherapy. However there is no suggestion that either of these agents will modulate Topo II. Furthermore the paper only describes the effect of these agents on apoptosis (studied by looking at the nuclei) and does not show synergy in terms of cell death or proliferation.

Blagosklonny et al. (Leukemia October 2001;15(10):1537-43) discloses that the ansamycin antibiotic (geldanamycin—an Hsp90 inhibitor) sensitises cells to the effects of Taxol or doxrubicin. There is no suggestion that any of the effects are to do with any interaction between HSP 90 and topoisomerase II.

Doxorubicin is known to have three potential mechanisms of action:
(a) Semiquinone free radical and oxygen radical generation;
(b) alteration of membrane fluidity and ion transport; and
(c). DNA intercalation-blocking synthesis of DNA and RNA.

A skilled person would consider the Munster and Blagosklonny papers to relate to the study of HSP90 modulated signalling pathways (i.e. RB- and Bcr-Abl pathways). They would consider the Munster and Blagosklonny papers and come to the conclusion that doxorubicin would not be effective in chemotherapy because it is modulating Topo II. This is because the state of the art dictates that HSP 90 inhibitors are effective in chemotherapy because they modulate signalling pathways and would not be expected to interact with Topo II (which is not a mediator of signal transduction).

Furthermore one skilled in the art would appreciate that both 17-AAG and doxorubicin are quinines and, as stated in a published commentary in the Munster paper (Sausville (2001) Clin Cancer Res Vol 7 2155-2158), would expect that there would be a high likelihood of the combination of these agents causing end-organ toxicity. Accordingly based on the prior art a skilled person would not chose to use these agents in combination because they would be toxic to an animal or human irrespective of what they may do to any tumour.

Several classes of compound may be used according to the invention as the first agent. These compounds include:
(i) compounds that bind to Topo II and inhibit its activity (e.g. competitive inhibitors; allosteric inhibitors or cleavable complex inhibitors);
(ii) compounds which prevent the transcription, translation or expression of Topo II (e.g. ribozymes or antisense DNA molecules e.g. antisense crossing the first intron/exon boundary);
(iii) compounds which inhibit release of Topo II from intracellular stores; and
(iv) compounds which increase the rate of degradation of Topo II.

Compounds may modulate human Topo IIα or Topo IIβ.

Doxorubicin is toxic and it is preferred that the first agent is not Doxorubicin.

A preferred class of first agents that may be used according to the invention are compounds that interfere with the breakage and religation of a G segment of DNA. Such compounds form structures that favour DNA strand breakage often referred to as “cleavable complexes.” In the absence of these compounds the cleavable complexes are usually short-lived whereas the presence of the preferred first agents induces a large number of cleavable complexes, which if unresolved ultimately lead to cell death. Examples of such first agents include:
Podophyllotoxin derivatives and analogues (e.g. etoposide);
Anthracenedione derivatives and analogues (e.g. Mitoxantrone); or
m-AMSA (amsacrine)

Amsacrine has the following properties:
m-AMSA
Amsidine
Amsine
Methanesulfonamide, N-[4-(9-acridinylamino)-3-methoxyphenyl]-(9CI)
WLN: T C666 BNJ IMR BO1 DMSW1
4′-(9-Acridinylamino)-3′-methoxymethanesulfonanilide 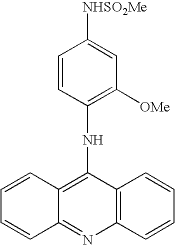
CAS Registry Number: 51264-14-3
NSC 249992

Bisdioxopiperazine derivatives (e.g. ICRF-154, 159, 187 & 193) represent a further class of first agents that may be used according to the invention. Bisdioxopiperazine derivatives inhibit DNA topoisomerases II by “locking” the ATP-operated clamp of the enzyme.

ICRF-187

ADR-529
Soluble ICRF (L-isomer)
2,6-Piperazinedione, 4,4′-(1-methyl-1,2-ethanediyl)bis-, (S)-(9CI)
   2,6-Piperazinedione, 4,4′-propylenedi-, (P)-(8CI)
      Chemical Data
CAS Registry Number: 24584-09-6 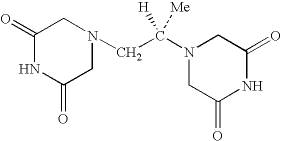
NSC 169780
   ICRF-159.
±.-(3,5,3′,5′-Tetraoxo)-1,2-dipiperazinopropane
   (.±.)-1,2-Bis(3,5-dioxopiperazinyl)propane
(±)-(3,5,3′,5′-Tetraoxo)-1,2-dipiperazinopropane
(±)-1,2-Bis(3,5-dioxopiperazinyl)propane
NSC 129943
Propane, (.±.)-1,2-bis(3,5-dioxopiperazin-1-yl)-Razoxin
2,6-Piperazinedione, 4,4′-(1-methyl-1,2-ethanediyl)bis-, (±)-(9CI)
2,6-Piperazinedione, 4,4′-(1-methyl-1,2-ethanediyl)bis-, (.±.)-(9CI)
2,6-Piperazinedione, 4,4′-propylenedi-2,6-Piperazinedione, 4,4′-propylenedi-, (.±.)-(8CI)
2,6-Piperazinedione, 4,4′-propylenedi-, (±)-(8CI) 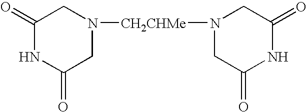
NSC 129943
CAS Registry Number 21416875

Thiobarbiturates (e.g. Merbarone or a derivative or analogue thereof); Genisten (or a derivative or analogue thereof); and Pyrazoloacridine (or a derivative or analogue thereof) represent further preferred first agents.

Merbarone

5-Pyrimidinecarboxamide, hexahydro-4,6-dioxo-N-phenyl-2-thioxo-CAS Registry Number 97534-21-9
http://dtp.nci.nih.gov/docs/static%5Fpages/pharm%5Fdata/336628.html 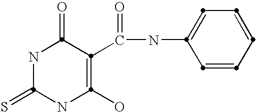
NSC 336628
CAS Registry Number: 97534-21-9
   Genisten
GENISTEIN
C.I. 75610 Genistein
Genisteol
Genisterin Isoflavone, 4′,5,7-trihydroxy- (8CI)
Prunetol
Sophoricol
4′,5,7-Trihydroxyisoflavone
4H-1-Benzopyran-4-one, 5,7-dihydroxy-3-(4-hydroxyphenyl)-(9CI) 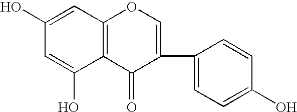
CAS Registry Number: 446-72-0
NSC 36586
   Pyrazoloacridine
Pyrazolo[3,4,5-kl]acridine-2(6H)-propanamine, 9-methoxy-N,N-dimethyl-5-nitro-, 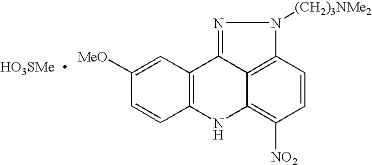
monomethanesulfonate
NSC 366140
CAS Registry Number: 99009-20-8

Most preferred first agents for use according to the invention are etoposide (VP16) and teniposide.

VP-16 (Etoposide)

DEMETHY-EPIPODOPHYLLOTOXIN,ETHYLIDENE GLUCOSIDE,
Epipodophyllotoxin VP-16213
Epipodophyllotoxin, 4′-demethyl-, 4,6-O-ethylidene-.beta.-D-glucopyranoside (8CI)
Epipodophyllotoxin, 4′-demethyl-, 9-(4,6-O-ethylidene-.beta.-D-glucopyranoside)
Etoposide
EPE
ETOPOSIDE
Furo[3′,4′:6,7]naphtho[2,3-d]-1,3-dioxol-6(5aH)-one, 9-[(4,6-O-ethylidene-.beta.-D-glucopyranosyl)oxy]-5,8,8a, 9-tetrahydro-5-(4-hydroxy-3,5-dimethoxyphenyl)-, [5R-[5.alpha., 5a.beta.,8a.alpha.,9.beta.(R*)]]-(9CI)
NSC 141540
NSC141540
Vepesid
Vepesid J
VP 16-213
VP-16
4-Demethylepipodophyllotoxin-.beta.-D-ethylideneglucoside
4′-Demethylepipodophyllotoxin ethylidene-.beta.-D-glucoside 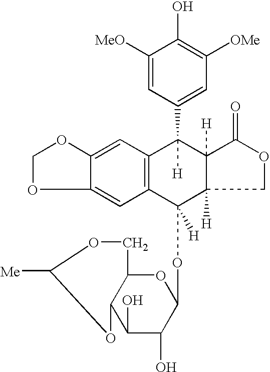
NSC 141540
CAS Registry Number: 33419420
   Teniposide
VM-26 (teniposide)
Epipodophyllotoxin, 4′-demethyl-, 9-(4,6-O-2-thenylidene-.beta.-D-glucopyranoside) (8CI) Epipodophyllotoxin, 4′-demethyl-, 9-(4,6-O-2-thienylidene-.beta.-D-glucopyranoside) (8CI)
EPT
Furo[3′,4′:6,7]naphtho[2,3-d]-1,3-dioxol-6(5aH)-one, 5,8,8a, 9-tetrahydro-5-(4-hydroxy-3,5-dimethoxyphenyl)-9-[[4,6-O-(2-thienylmethylene)-.beta.-D-glucopyranosyl]oxy]-, [5R-[5.alpha.,5a.beta.,8a.alpha.,9.beta.(R*)]]-(9CI)
Furo[3′,4′:6,7]naphtho[2,3-d]-1,3-dioxol-6(5aH)-one, 5,8,8a, 9-tetrahydro-5-(4-hydroxy-3,5-dimethoxyphenyl)-9-[[4,6-O-(2-thienylmethylene)-.beta.-D-glucopyranosyl]oxy]-, [5R-(5.alpha.,5a.beta.,8a.alpha.,9.beta.)]-
Furo[3′,4′:6,7]naphtho[2,3-d]-1,3-dioxol-6(5aH)-one, 5,8,8a, 9-tetrahydro-5-(4-hydroxy-3,5-dimethoxyphenyl)-9-[[4,6-O-(2-thienylmethylene)-.beta.-D-glucopyranosyl]oxy]-, [5R-[5.alpha.,5a.beta.,8a.alpha.,9.beta.(R*)]]-(9CI)
Furo[3′,4′:6,7]naphtho[2,3-d]-1,3-dioxol-6(5aH)-one, 5,8,8a, 9-tetrahydro-5-(4-hydroxy-3,5-dimethoxyphenyl)-9-[[4,6-O-(2-thienylmethylene)-.beta.-D-glucopyranosyl]oxy]-, [5R-(5.alpha.,5a.beta.,8a.alpha.,9.beta.)]-
NSC 12819
NSC122819
PTG
Teniposide
TENIPOSIDE
Veham-Sandoz
Vehem
Vumon
VM 26
4-Demethylepipodophyllotoxin-.beta.-D-thenylideneglucoside
4′-Demethylepipodophyllotoxin thenylidene glucoside 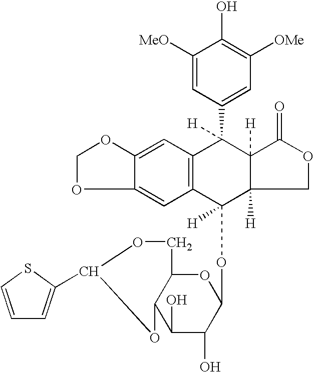
NSC 122819
CAS Registry Number: 29767202

Human Topoisomerase II isoforms are known to interact with Sgs1p (a eukaryotic homolog of E. Coli RecQ) and Pat1 (a 90 kDa pro line and glutamine rich protein). These proteins are required, together with Topoisomerase II, to affect chromosome segregation and have been isolated using the yeast two hybrid cloning system. Additionally, a physical association between Topoisomerase II and the underphosphorylated form of Rb protein has been established by reciprocal immunoprecipitation and immunoblotting. Numerous other proteins have also been shown to interact with topoisomerase II including; cAMP response element protein, ATF-2, Jun, CD3ε, Barren, small ubiquitin-related modifier-1, caspase-activated DNase and histone deacetylases. It will be appreciated that agents which modulate such proteins may also be used as first agents according to the invention.

Several classes of compound may be used according to the invention as the second agent. These compounds include:
(i) compounds that bind to HSP90 and inhibit its activity (e.g. competitive inhibitors or allosteric inhibitors);
(ii) compounds which prevent the transcription, translation or expression of HSP90 (e.g. ribozymes or antisense DNA molecules);
(iii) compounds which inhibit release of HSP90 from intracellular stores; and
(iv) compounds which increase the rate of degradation of HSP90.

Geldanamycin and its derivatives (e.g. 17-Allylamino, 17-demethoxygeldanamycin-17-AAG or Macbecin II) are preferred second agents for use according to the present invention. These include:

Macbecin II

Geldanamycin, 18,21 -didehydro-6,17-didemethoxy-18,21-dideoxo-18,21 -dihydroxy-15-methoxy-6-methyl-11-O-methyl-, (6S,15R)-(9CI)
Geldanamycin, 18,21 -didehydro-6,17-didemethoxy-18,21-dideoxo-18,21 -dihydroxy-15-methoxy-6-methyl-11-O-methyl-
Geldanamycin, 18,21-didehydro-6,17-didemethoxy-18,21-dideoxo-18,21-dihydroxy-15-methoxy-6-methyl-11-o-methyl-, (6S,15R)-
MACBECIN II 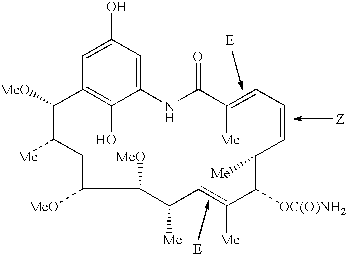
2-Azabicyclo[16.3.1]docosane, geldanamycin deriv. (9CI)
NSC 330500
CAS Registry Number: 73341738
   Geldanamycin 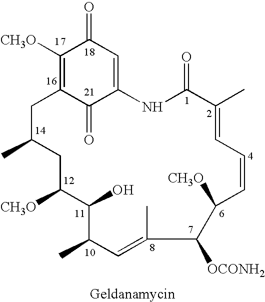
NSC 122750

Radicicol may be used as a second agent according to the invention.
Radicicol

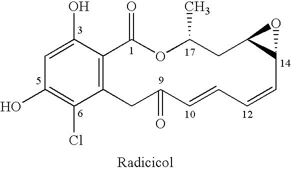

It will be appreciated that agents may be developed that have a dual action in that they are able to attenuate Topo II activity and also inhibit Hsp90. Such agents may be used in an adaption of the present invention which involves the use of a single, dual action, agent only rather than separate first and second agents. An example of such an agent is an agent that bind to the ATPase domain of both Topo II and HSP90. An example of such agents include quinilone derivatives (see below) and also novobicin. Novobicin is particularly useful for treating micro organisms.

The first and second agents may be further combined with other therapeutics when there is a medical need. For instance, for certain medical conditions, the inventor has found even greater therapeutic efficacy when the agents are combined with a mediacment which suppresses apoptosis in non-cancerous tissue(eg pifithrin-α).

Agents which attenuate Topo II activity and inhibit Hsp90 may be used in chemotherapy to treat a number of conditions requiring the induction of targeted cell death. These include:
1) Cancer chemotherapy;
2) antibacterial treatments;
3) antifungal treatments;
4) the treatment of AIDS/HIV;
5) the treatment of multiple sclerosis; and
6) the killing and inhibition of proliferation of any organism.

When used to treat cancer, the agents are particularly effective for treating solid tumours such as bowel cancer, small cell and non-small cell lung cancer, head and neck cancer, breast cancer, bladder cancer and malignant melanoma.

The combined agents are also particularly effective for the treatment of paediatric tumours such as neuroblastoma and in the treatment of leukaemias and lymphomas, in which both proteins are contemporaneously or sequentially targeted.

The inventors have found that the combination of first and second agents improves the effectiveness of the agents for all known clinical applications for the agents. For instance, combination of etoposide (VP16) with an Hsp90 inhibitor synergistically improves the effectiveness of etoposide for treating:
Adult Acute Myeloid Leukemia
Adult Hodgkin's Disease
Adult Non-Hodgkin's Lymphoma
AIDS-Related Lymphoma
Carcinoma of Unknown Primary
Childhood Acute Myeloid Leukemia
Childhood Brain Tumor
Childhood Cerebral Astrocytoma
Childhood Ependymoma
Childhood Hodgkin's Disease
   Childhood Liver Cancer
   Childhood Medulloblastoma
   Childhood Non-Hodgkin's Lymphoma
   Childhood Rhabdomyosarcoma
   Childhood Supratentorial Primitive Neuroectodermal and Pineal Tumors
   Childhood Visual Pathway and Hypothalamic Glioma
   Endometrial Cancer
   Ewing's Family of Tumors Including Primitive Neuroectodermal Tumor (PNET)
   Extragonadal Germ Cell Tumors
   Gastric Cancer
   Gastrointestinal Carcinoid Tumor
   Gestational Trophoblastic Tumor
   Kaposi's Sarcoma
   Malignant Thymoma
   Neuroblastoma
   Non-small Cell Lung Cancer
   Osteosarcoma/Malignant Fibrous Histiocytoma of Bone
   Ovarian Epithelial Cancer
   Ovarian Germ Cell Tumor
   Pediatric Extracranial Germ Cell Tumor
   Prostate Cancer
   Retinoblastoma
   Small Cell Lung Cancer
   Testicular Cancer
   Unusual Cancers of Childhood
   Wilms' Tumor and Other Childhood Kidney Tumors

When the agents are used to treat non-mammalian organisms, or to attack micro-organisms, it is preferred that the agents are effective for attenuating the activity of the species equivalent of Topo II or inhibiting the species equivalent of HSP90. For instance, when the agents are used as antibacterial agents it is preferred that they attenuate the activity of DNA gyrase (a bacterial type of topoisomerase II) and inhibit pHtpG (a bacterial equivalent of HSP 90).

We have found that the agents are particularly useful for arresting the growth or directly killing a number of bacteria. These include gram −ve and gram +ve bacterium.

The agents may be used to treat a number of bacterial infections in mammals (and particularly humans). Bacteria that may be attacked according to the invention are listed below. The conditions caused by such bacteria, and thereby treatable by the combination therapy according to the invention, are indicated in parentheses.

The following bacteria may be treated according to the invention:
Abiotrophia (Reported infections—endophthalmitis, brain abscess, osteomyelitis);
Achromobacter (Reported infections—septicaemia, CAPD peritonitis, pneumonia, ear infection);
Acidaminococcus (Reported infections—abscesses, post surgical infections);
Acidovorax (Reported infections—wound infection, UTI, bacteraemia, meningitis, septic arthritis);
Acinetobacter (Reported infections—septicaemia, UTI, wound infections abscesses, endocarditis, meningitis, osteomyelitis);
Actinobacillus (Reported infections—periodontitis, endocarditis, abscesses, pericarditis, meningitis, septicaemia, pneumonia, empyema, hepatitis);
Actinobaculum (Reported infections—pyelonephritis);
Actinomadura (Reported infections—actinomycetoma, madura foot);
Actinomyces (Reported infections—actinomycosis);
Aerococcus (Reported infections—endocarditis, UTI, wound infection, meningitis, abscesses);
Aeromonas (Reported infections—wound infection, abscesses, septicaemia, acute diarrhoea, meningitis, leech bite infection, alligator bite infection, infections associated with aquatic exposure);
Afipia (Reported infections—cat scratch disease (A. felis), septic arthritis, bone marrow infection (A. broomeae), bone infection (A. clevelandensis);
Agrobacterium (Reported infections—endocarditis,CAPD peritonitis, UTI, line sepsis);
Alcaligenes (Reported infections—pneumonia, otitis, UTI, osteomyelitis, bacteraemia);
Alloiococcus (Reported infections—otitis media);
Amycolata (Please see Pseudonocardia);
Amycolatopsis (Species associated with infection—A. orientalis);
Anaerobospirillum (Reported infections—diarrhoea, bacteraemia);
Anaerorhabdus (Reported infections—lung abscess, appendix absecess, abdominal abscess);
“Anguillina” (Species associated with infection—“Anguillina coli”);
Arachnia (Species associated with infection—Arachnia propionica);
Arcanobacterium (Reported infections—septic arthritis (A. bernardiae and A. pyogenes), UTI and septicaemia (A. bernardiae), tonsillitis, cellulitis, lymphadenitis, brain abscess,septicaemia, osteomyelitis (A. haemolyticum);
Arcobacter (Reported infections—enteric infection (diarrhoea and abdominal cramps);
Arthrobacter (Reported infections—UTI, bacteraemia, Whipple's disease);
Atopobium (Reported infections—UTI, dental abscess, pelvic abscesses, wound infection);
Aureobacterium (With the exception of A. resistens, Aureobacterium spp. have been reclassified as members of the genus Microbacterium. The name A. resistens (which is vancomycin-resistent) was validly after other Aureobacterium spp. were reclassified as Microbacterium spp. Aureobacterium isolates have been misidentified as “Corynebacterium aquaticum”);
Bacillus (B. anthracis—the agent of anthrax B. thuringiensis, a biological insecticide has caused corneal infection);
Bacteroides (Reported infections—abscesses, bacteraemia, bite infections, wound infections, chronic otitis media, pelvic inflammatory disease);
Balneatrix (Reported infections—pneumonia, bacteraemia, meningitis);
Bartonella (Reported infections—Oroya fever and verruga peruana (B. bacilliformis), cat scratch disease (B. henselae), bacillary angiomatosis (B. henselae, B. guintana), trench fever (B. quintana), endocarditis (B. elizebethae), bacteraemia (B. vinsonii arupensis);
Bergevella (Reported infections—wound infection, septicaemia, meningitis);
Bifidobacterium (Reported infections—bacteraemia, peritonitis, abscesses, otitis, paronychia);
Bilophila (Reported infections—appendicitis, abscesses, bacteraemia, biliary tract sepsis);
Branhamella (Species associated with infection—B. catarrhalis, this organism has been reclassified as Moraxella catarrhalis);
Borrelia (Species associated with relapsing fever, Reported infections—wound infection, septicaemia, meningitis);
Bordetella (Reported infections—respiratory tract infection (B. bronchiseptica, B. parapertussis, B. pertussis), whooping cough (B. parapertussis, B. pertussis), bacteraemia, otitis, wound infection (B. hinzii, B. holmseii, B. trematum);
Brachyspira (Reported infections—intestinal spirochaetosis);
Brevibacillus (Reported infections—endophthalmitis, food poisoning, bacteraemia);
Brevibacterium (Reported infections—bacteraemia, meningitis, chest infection);
Brevundimonas (Reported infections—septicaemia);
Brucella (Reported infections—brucellosis);
Burkholderia (associated infections include lung infection, bacteraemia, endocarditis, septic arthritis, UTI, cystic fibrosis patients);
Buttiauxella (Reported infections—appendicitis, wound infection);
Butyrivibrio (Reported infections—endophthalmitis);
Calymmatobacterium (This taxon has been reclassified as Klebsiella granulomatis);
Campylobacter (associated with diarrhoea, bacteraemia, periodontitis, appendicitis, peritonitis and head and neck infections fever, meningoencephalitis, endocarditis, abscesses and abscess—zoonoses from mammals and birds, Campylobacter butzleri was reclassified as Arcobacter butzleri, C. cinaedi, C. fennelliae, C. pyloridis were reclassified as Helicobacter spp);
Capnocytophaga (wound infection, septicaemia, abscesses, meningitis, endocarditis—associated with dog bites systemic infections in neutropenic patients);
Cardiobacterium (Reported infections—endocarditis, meningitis);
Catonella (Reported infections—periodontitis);
Cedecea (Reported infections—bacteraemia);
Cellulomonas (reported cases of bacteraemia, meningitis cases of bacteraemia, endocarditis);
Centipeda (Reported infections—periodontitis);
Chlamydia (Reported infections—trachoma, genital infection, neonatal infection, lymphogranuloma venereum);
Chlamydonhila (associated with abortion following contact with infected ruminants, associated with chest infection agent of psittacosis, a zoonosis from birds);
Chromobacterium (Reported infections—septicaemia, osteomyelitis, abscesses, eye infection);
Chyseobacterium (Reported infections—bacteraemia, meningitis, abdominal sepsis, wound infection, line infection);
Chryseomonas (Chryseomonas luteola has been reclassified as Pseudomonas luteola);
Citrobacter (Reported infections—UTI, meningitis, haemolytic-uraemic syndrome);
Clostridium (associated with wound infection, bacteraemia and abscesses, botulism, diarrhoea (usually antibiotic-associated) and pseudomembranous colitis, food poisoning, necrotising enterocolitis (pigbel, Darmbrand), gas gangrene—(C. histolyticum, C. novyi, C. septicum, C. sordellii also associated with gas gangrene), tetanus);
Collinsella (Species associated with infection—Collinsella aerofaciens);
Comamonas (Reported infections—bacteraemia, conjunctivitis);
Corynebacterium (associated with infections such as septicaemia, peritonitis, eye infection, wound infection, endocarditis, osteomyelitis, septic arthritis, meningitis and abscesses diphtheria and cutaneous infection, tropical ulcer, septicaemia, pulmonary infection, lymphadenitis pharyngitis or diphtheria-like illness);
Coxiella (The agent of Q fever);
Cryptobacterium (Associated with periodontitis);
Delftia (Reported cases of bacteraemia and endocarditis);
Dermabacter (brain abscess, bacteraemia, wound infection);
Dermatophilus (Reported to cause cutaneous infection—zoonosis from cattle, sheep, goats and horses);
Desulfomonas (Associated with pilonidal abscess and peritonitis);
Desulfovibrio (Reported infections—bacteraemia, liver abscess);
Dialister (Reported infections—periodontitis);
Dichelobacter (Reported infections—pilonidal cyst, rectal fistula, wound infection);
Dolosicoccus (Reported infections—bacteraemia);
Dolosigranulum (Reported infections—spinal cord infection, eye infection);
Edwardsiella (Reported infections—wound infections, abscesses, gastroenteritis—associated with aquatic exposure and penetrating fish injury);
Eggerthella (Reported infections—rectal abscess);
Ehrlichia (Reported infections—Ehrlichiosis);
Eikenella (Reported infections—septicaemia, endocarditis, abscesses, septic arthritis);
Empedobacter (Species associated with infection—E. brevis);
Enterobacter (Associated infections—bacteraemia, respiratory tract infections, UTI—associated with nosocomial infection);
Enterococcus (Associated infections—bacteraemia, abscesses, endocarditis, meningitis, UTI, peritonitis, osteomyelitis, wound infection);
Erwinia (Associated infections—UTI);
Erysipelothrix (Associated infections—erysipeloid, septicaemia, endocarditis);
Escherichia (associated with UTI, bacteraemia, wound infection, meningitis, enteric infection, haemolytic uraemic syndrome);
Eubacterium (Associated infections—wound infection, abscesses, septicaemia, periodontitis);
Ewingella (Associated infections—septicaemia, wound infection, UTI);
Exiguobacterium (Species associated with infection—E. acetyliticum, E. aurantiacum);
Facklamia (Associated infections—UTI, bacteraemia, abscess);
Filifactor (Associated infections—gingivitis, periodontitis);
Flavobacterium (Associated infections—bacteraemia, diarrhoea);
“Flexispira” (Associated infections—bacteraemia, diarrhoea);
Francisella (associated with septicaemia and invaxsive systemic infection, tularaemia);
Fusobacterium (Associated infections—abscesses, bacteraemia, periodontitis, endocarditis, necrobacillosis);
Gardnerella (Associated infections—intrauterine and neonatal sepsis—associated with bacterial vaginosis);
Gemella. (Associated infections—bacteraemia, endocarditis);
Globicatella (Associated infections—bacteraemai, UTI, meningitis);
Gordona (Associated infections—pulmonary infection, sternal wound sepsis, brain abscess, bacteraemia);
Haemophilus (associated with Brazilian purpuric fever; associated with sinusitis, otitis media, pneumonia, abscesses, endocarditis; the agent of chancroid; associated with bacteraemia, meningitis, epiglottitis, respiratory tract infection);
Hafnia (Associated infections—bacteraemia—has been associated with cases of diarrhoea);
Helicobacter (a zoonosis from dogs and hamsters cause of gastroenteritis; associated with septiciaemia and proctitis; sepicaemia in a neonate; gastritis);
Helococcus (associated with sebaceous cyst infection and breast absecess);
Holdemania (Species associated with infection—H. filiformis);
Ignavigranum (Associated infections—wound infection, ear abscess);
Johnsonella (Associated infections—perodontitis);
Kingella (Associated infections—septic arthritis, endocarditis);
Klebsiella (associated with UTI, bacteraemia, wound infection, respiratory tract infection; rhinoscleroma);
Kocuria (Species associated with infection—K. varians, K. kristinae);
Koserella (Associated infections—wound infection, septic arthritis);
Kurthia (bacteraemia and endocarditis; diarrhoea);
Kytococcus (Species associated with infection—K. sedentarius);
Lactobacillus (Associated infections—abscesses, bacteraemia, endometritis, endocarditis, lung infection, UTI—reported risk factors for infection, surgery, malignacy, diabetes mellitus, immunodeficiency);
Lactococcus (Associated infections—bacteraemia, endocarditis, UTI);
Lautropia (has been isolated from oral flora of an HIV patient and from sputum of a cystic fibrosis patient);
Leclercia (Associated infections—bacteraemia, wound infection);
Legionella (Associated infections—legionaires' disease, Pontiac fever);
Leminorella (Associated infections—UTI);
Leptosipira (Associated infections—leptospirosis);
Leptotrichia (Associated infections—bacteraemia, endocarditis);
Leuconostoc (Associated infections—meningitis, bacteraemia, pulmonary infection);
Listeria (Associated infections—septicaemia, meningitis, intra-uterine infection, enteric infection);
Megasphaera (Associated infections—septicaemia, meningitis, intra-uterine infection, enteric infection);
Methylobacterium (Associated infections—bacteraemia, CAPD peritonitis);
Microbacterium (Associated infections—endophthalmitis, UTI, endocarditis, soft tissue infection, hypersensitivity pneumonitis, meningitis, CAPD peritonitis);
Micrococcus (Associated infections—bacteraemia, endocarditis, septic arthritis);
Mitsuokella (Species associated with infection—M. multiacida);
Mobiluncus (Associated infections—endometritis, chorioamnionitis—associated with bacterial vaginosis);
Moellerella (diarrhoea);
Moraxella (associated with—conjunctivitis, wound infection, endocarditis, abscesses, osteomyelitis);
Morganella (Associated infections—bacteraemia, UTI, wound infection);
Mycobacterium (Leprosy, cervical adenitis, Buruli ulcer, fish-tank granuloma, M. malmoense, M. szulgai, M. kansasii, M. xenopi—associated with pulmonary infection, systemic infection in imunocompromised patients, post-inoculation infection);
Mycoplasma (Associated infections—respiratory infection, post-partum fever, pyelonephritis, pelvic inflammatory disease, myocarditis, pericarditis, meningitis);
Myroides (Associated infections—UTI, wound infection);
Neisseria (associated with meningitis, bacteraemia, endocarditis, osteomyelitis, agent of genital gonorrhoea, septicaemia, ophthalmia neonatorum, associated with septicaemia, meningitis, conjunctivitis, genital infection, epiglottitis)
Nocardia (nocardiosis);
Nocardiopsis (Associated infections—mycetoma, cutaneous infection, pulmonary infection, conjunctivitis);
Ochrobactrum (Associated infections—bacteraemia, endophthalmitis, liver abscess—reported association with nosocomial infections in debilitated patients);
Oeskovia. (associated with meningitis, pyelonephrosis, CAPD peritonitis, endophthalmitis);
Oligella (associated with UTI, septicaemia—infection associated with urinary catheters);
Orientia (Associated infections—scrub typhus);
Paenibacillus (Associated infections—septicaemia, meningitis, pneumonia);
Pantoea (Associated infections—bacteraemia, endocarditis, wound infection, cellulitis, alligator bite infection, endophthalmitis);
Parachlamydia (Parachlamydia acanthamoebae has been associated with hypersensitivity pneumonitis (humidifier fever);
Pasteurella (Associated infections—wound infection, septicaemia, abscesses, pneumonia, endocarditis, meningitis—infections relate to spp);
Pediococcus (Associated infections—bacteraemia, abscesses, pulmonary infection—infections in debilitated patients);
Peptococcus (Peptococcus niger has been associated with anaerobic infections including intra-abdominal sepsis);
Photobacterium (Associated with necrotising wound infection);
Photorhabdus (Associated infections—bacteraemia, wound infection);
Plesiomonas (Associated infections—gastroenteritis, septicaemia, meningitis, endophthalmitis);
Porphyrimonas (Associated infections—mixed anaerobic infections at various sites, periodontitis, associated with bite infections (human and animal);
Prevotella (Associated infections—abscesses, bacteraemia, wound infection, bite infections, genital tract infections, periodontitis);
Propionibacterium (Associated infections—abscesses, endocarditis, bacteraemia, spetic arthritis, endophthalmitis, acne vulgaris);
Proteus (Associated infections—UTI, bacteraemia, wound infection, abscesses);
Providencia. (Associated infections—UTI, wound infection, bacteraemia);
Pseudomonas (Reported infections—bacteraemia, UTI, wound infection, abscesses, septic arthritis, conjunctivitis, endocarditis, meningitis, CAPD peritonitis—nosocomial)
Pseudonocardia (Species associated with infection—P. autotrophica)
Pseudoramibacter (Associated infections—periodontal disease, wound infection, abscesses)
Psychrobacter (Associated infections—meningitis, bacteraemia, eye infection);
Rahnella (Associated infections—UTI, septicaemia);
Ralstonia (Associated infections—bacteraemia, UTI, meningitis, wound infection, peritonitis);
Rhodococcus (associated with bacteraemia, osteomyelitis, lung abscesses—infections of immunocompromised patients including AIDS);
Rickettsia (Associated infections—rickettsial spotted fever, tick typhus, tick bite fever, rickettsialpox);
Roseomonas (Associated infections—bacteraemia, wound infection, peritonitis);
Rothia (Associated infections—endocarditis, abscesses);
Ruminococcus (Associated infections—abdominal sepsis, abscesses);
Salmonella (Associated infections—gastroenteritis, enteric fever, osteomyelitis);
Selenomonas (Associated infections—bacteraemia, lung abscess—infections reported to be associated with malignancy or alcohol abuse);
Serratia (Associated infections—septicaemia, abscesses, burn infections, osteomyelitis);
Shewenella (associated with cases of intra-abdominal sepsis, meningitis and bacteraemia);
Shigella (Associated infections—enteric infection);
Simkania (Associated infections—bronchiolitis, pneumonia);
Slackia (Associated infections—periodontitis);
Sphingobacterium (Associated infections—bacteraemia, UTI, peritonitis);
Sphingomonas (Associated infections—septicaemia, UTI, wound infections, CAPD peritonitis—nosocomial infections);
Spirillum (Associated infections—rat bite fever);
Staphylococcus (Associated infections—Bacteraemia, wound infection, endocarditis, catheter-related sepsis,UTI, toxic shock syndrome, eye infection, osteomyelitis);
Stenotrophomonas (associated with various (mostly nosocomial) infections—bacteraemia, meningitis, wound infection, UTI and pneumonia);
Stomatococcus (Associated infections—endocarditis, meningitis, neutropenic sepsis);
Streptobacillus (Associated infections—rat bite fever, Haverhill fever);
Streptococcus (Associated infections—pharyngitis, bacteraemia, pyogenic infection, necrotising infection, septic arthritis, glomerulonephritis, meningitis, rheumatic fever, abscesses, endocarditis, pharyngitis, wound infection, pneumonia, pericarditis, CAPD, peritonitis, sinusitis, otitis, conjunctivitis);
Streptomyces (Associated infections—actinomycetoma);
Succinivibrio (Associated infections—bacteraemia);
Sutterella (Associated infections—appendicitis, peritonitis, abascesses, osteomyelitis);
Suttonella (Associated infections—endocarditis, eye infection);
Tatumella (Associated infections—bacteraemia, UTI);
Tissierella (Associated infections—bacteraemia);
Trabulsiella (Associated infections—diarrhoea);
Treponema (associated with periodontal disease, pinta, genital lesions, venereal and non-venereal endemic syphilis, yaws);
Tropheryma (associated with Whipple's disease);
Turicella (Associated infections—otitis, cervical abscess);
Ureaplasma (Associated infections—urethritis);
Vagococcus (Species associated with infection—V. fluvialis).
Veillonella (Associated infections—abscesses, bacteraemia);
Vibrio (The agent of cholera, associated with wound infection, bacteraemia, diarrhoea and septiciaemia, septicaemia, meningitis, endometritis);
Weeksella (associated with peritonitis and vaginal infections);
Xanthomonas (bacteraemia);
Yersinia (agent of plague, associated infections—enterocolitis, soft tissue infections, mesenteric lymphadenitis, enteric infection);
Yokenella (Associated infections—bacteraemia, wound infection);

The inventors have found that the quinilone derivatives listed below are particularly useful as first agents according to the invention when used to attack bacteria.

Quinolones—family

nalidixic acid [NegGram^{R}]
Cinoxacin [Cinobac^{R}]
Oxolinic acid [UtiBID^{R}]
   FLUOROquinolones
Ciprofloxacin [Cipro^{R}]
Clinafloxacin Enoxacin [Penetrex^{R}]
Lomefloxacin [Maxaquin^{R}]
Norfloxacin [Noroxin^{R}]
Ofloxacin [Floxin^{R}, Floxin IV^{R}]
Levofloxacin [Levaquin^{R}] (L-isomer of ofloxacin)
Sparfloxacin [Zagam^{R}]
Grepafloxacin [Raxar^{R}]
Gatifloxacin [TequinR]
Moxifloxacin hydrochloride [Avelox^{R}]
Trovafloxacin mesylate [Trovan^{R} tablets]
Alatrofloxacin mesylate [Trovan^{R} I.V.]
Fleroxacin
Perfloxacin
Amifloxacin

The combination therapy may also be used to treat fungal infections of a subject. The agents are effective against the following fungi:
Candida spp, Aspergillus spp. Malassezia spp, Trichosporon spp, Fusarium spp, Paecilomyces spp and Acremonium spp, also Rhizopus, Mucor, Absidia, Blastomyces spp, Coccidiodes spp, Cryptococcus spp, Histoplasma spp

The inventors have found that the combination therapy is particularly useful for treating infection (eg local or systemic or deep systemic infections) associated with immune suppressed patients; urinary tract, bloodstream infections and pneumonia.

The agents may be used to treat existing medical conditions but may also be used when prophylactic treatment is considered medically necessary.

The agents used according to the invention may take a number of different forms depending, in particular on the manner in which they are to be used. Thus, for example, the agents may be in the form of a powder, tablet, capsule, liquid, ointment, cream, gel, hydrogel, aerosol, spray, micelle, liposome or any other suitable form that may be administered to a person or animal. It will be appreciated that the vehicle for the agents should be one which is well tolerated by the subject to whom it is given and enables delivery of the agent to the target tissue.

The agents may be used in a number of ways. For instance, systemic administration may be required in which case the agents may be contained within a composition which may, for example, be ingested orally in the form of a tablet, capsule or liquid. Alternatively the agents may be administered by injection into the blood stream. Injections may be intravenous (bolus or infusion) or subcutaneous (bolus or infusion). The compounds may also be administered by inhalation (e.g. intranasally).

The agents may also be incorporated within a slow or delayed release device. Such devices may, for example, be inserted under the skin and the compound may be released over weeks or even months. The devices may be particularly advantageous when an agent is used which would normally require frequent administration (e.g. at least daily ingestion of a tablet or daily injection).

It is preferred that second agents according to the invention are initially dissolved in solvents such as DMSO before dilution in aqueous solution for the preparation of liquid medicaments.

The agents may be formulated as prodrugs. Such prodrugs may be stored as inactive and stable medicaments which are subsequently activated.

It will be appreciated that the amount of an agent required is determined by biological activity and bioavailability that in turn depends on the mode of administration and the physicochemical properties of the agents employed. The frequency of administration will also be influenced by the abovementioned factors and particularly the half-life of the agents within the subject being treated.

Known procedures, such as those conventionally employed by the pharmaceutical industry (e.g. in vivo experimentation, clinical trials etc), may be used to establish specific formulations of agents and precise therapeutic regimes (such as daily doses and the frequency of administration).

Generally, a daily dose of between 0.01 μg/kg of body weight and 1.0 g/kg of body weight of a first agent and a second agent may be used for chemotherapy depending upon which specific agents are used. More preferably the daily dose of each agent is between 0.1 μg/kg of body weight and 100 mg/kg of body weight.

Purely by way of example suitable doses of first agents according to the invention are:
(a) A suitable dose of Amsacrine and derivatives thereof for treating a human cancer is 1 μg-1 g/M² IV (depending upon the health status of the individual). It is preferred that between 450 mg/M² IV and 600 mg/M² IV is given to a person over 3-5 days.
(b) A suitable dose of Etoposide, and all podophyllotoxin derivatives and analogues, for treating a human cancer is 1 μg-1 g/M2 IV (depending upon the health status of the individual). It is preferred that between 60 mg/M² IV and 120 mg/M² IV is given to a person daily for 5 consecutive days.
(c) A suitable dose of Mitoxantrone, and all anthracenedione derivatives and analogues, for treating a human cancer is 1 μg-1 g/M² IV (depending upon the health status of the individual). It is preferred that between 12 mg/M² IV and 14 mg/M² IV is given to a person every 21 days.
(d) When the first agent is Merbarone (or a derivative or analogue thereof); Genisten (or a derivative or analogue thereof); Pyrazoloacridine (or a derivative or analogue thereof); or ICRF 154, 159, 187 and 193 (or derivatives and analogues thereof) a preferred dose is 1 μg-1 g/M².
(e) A suitable dose of quinilone first agents (which are particularly useful for treating bacterial infections) is to give a human about 1-10,000 mg/day. It will be appreciated that the precise dose will depend upon the specific infection being treated.

Purely by way of example suitable doses (for cancer chemotherapy or the treatment of micro organisms) of second agents according to the invention are:
(a) A suitable dose of Radicicol (or a derivative or analogue thereof) for treating a human cancer is 1 ng-1g/M² (depending upon the health status of the individual).
(b) A suitable dose of Geldanamycin for treating a human cancer is 1 ng-1 g/M².
(c) A suitable dose of 17-AAG for treating a human cancer is 1 ng-1 g/M².

For all agents it is preferred that about 1 μg-1 g/kg of a first or a second agent is used for vetinary purposes. For instance about 4-25 mg/kg of Geldanamycin may be used.

Daily doses may be given as a single administration (e.g. a daily tablet for oral consumption or as a single daily injection). Alternatively the agents used may require administration twice or more times during a day. A patient receiving treatment may take a first dose upon waking and then a second dose in the evening (if on a two dose regime) or at 3 or 4 hourly intervals thereafter. Alternatively a slow release device may be used to provide optimal doses to a patient without the need to administer repeated doses. A preferred route of administration is by intravenous infusion. Administration may be over several hours or even days.

A preferred means of using protein or peptide agents is to deliver such agents to the target tissue by means of gene therapy. For instance, gene therapy may be used to decrease expression of Topo II or HSP90, decrease expression of enzyme(s) responsible for the intracellular synthesis of Topo II or HSP90, increase expression of a protein which promotes breakdown of Topo II or HSP90. Therefore according to a fourth aspect of the present invention there is provided a delivery system for use in a gene therapy technique, said delivery system comprising:
(i) a first DNA molecule encoding for a protein which directly or indirectly attenuates Topoisomerase II activity; and
(ii) a second DNA molecule encoding for a protein which directly or indirectly inhibits Heat Shock Protein 90 activity;
wherein said DNA molecules are capable of being transcribed to allow the expression of said proteins and thereby be effective for chemotherapy.

The delivery systems according to the fourth aspect of the invention are highly suitable for achieving sustained levels of a protein which are chemotherapeutically active over a longer period of time than is possible for most conventional therapeutic regimes. The delivery system may be used to induce continuous protein expression from cells in a target tissue that have been transformed with the DNA molecule. Therefore, even if the proteins have a very short half-life as agents in vivo, therapeutically effective amounts of the proteins may be continuously expressed from the treated tissue.

Furthermore, the delivery system of the invention may be used to provide the DNA molecules (and thereby the proteins which are active therapeutic agents) without the need to use conventional pharmaceutical vehicles such as those required in tablets, capsules or liquids.

The delivery system of the present invention is such that the DNA molecules are capable of being expressed (when the delivery system is administered to a patient) to produce proteins that directly or indirectly have activity for attenuating Topoisomerase II activity and inhibiting Heat Shock Protein 90 activity. By “directly” we mean that the product of gene expression per se has the required activity. By “indirectly” we mean that the product of gene expression undergoes or mediates (e.g. as an enzyme) at least one further reaction to provide an agent effective for attenuating Topoisomerase II activity or inhibiting Heat Shock Protein 90 activity.

The DNA molecules may be contained within a suitable vector to form a recombinant vector. The vector may for example be a plasmid, cosmid, virus or phage. Such recombinant vectors are highly useful in the delivery systems of the invention for transforming cells with the DNA molecule.

The recombinant vector may also further comprise a promoter or regulator to control expression of the gene as required.

It will be appreciated that the first and second DNA molecules may be contained within a single vector and the expression thereof may be driven from either a single promoter or individual promoters. Alternatively the delivery system may comprise first and second DNA molecules contained within respective first and second expression vectors.

Recombinant vectors may also include other functional elements. For instance, recombinant vectors can be designed such that the vector will autonomously replicate in the cell. In this case, elements that induce DNA replication may be required in the recombinant vector. Alternatively the recombinant vector may be designed such that the vector and recombinant DNA molecule integrates into the genome of a cell. In this case DNA sequences which favour targeted integration (e.g. by homologous recombination) are desirable. Recombinant vectors may also have DNA coding for genes that may be used as selectable markers in the cloning process.

The DNA molecules may (but not necessarily) be one which becomes incorporated in the DNA of cells of the subject being treated. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells in the subject being treated. When this is the case, regulation of expression may be less important because expression of the DNA molecules will stop when the transformed cells die or stop expressing the proteins (ideally when chemotherapy is no longer required).

The delivery system may provide the DNA molecules to the subject without them being incorporated in a vector. For instance, the DNA molecules may be incorporated within liposomes or virus particles. Alternatively the “naked” DNA molecules may be inserted into a subject's cells by a suitable means e.g. direct endocytotic uptake.

The DNA molecules may be transferred to the cells of a subject to be treated by transfection, infection, microinjection, cell fusion, protoplast fusion or ballistic bombardment. For example, transfer may be by ballistic transfection with coated gold particles, liposomes containing the DNA molecules, viral vectors (e.g. adenovirus) and means of providing direct DNA uptake (e.g. endocytosis) by application of the DNA molecules directly to the target tissue topically or by injection.

The discovery that Topo II and HSP90 interact has enabled the inventor to develop a drug screening assay system for testing the efficacy of candidate drugs as chemotherapeutic agents. Therefore the two interacting proteins HSP90 and Topoisomerase II may be used as a complex target for new drug development in which both proteins are contemporaneously or sequentially targeted for new mammalian, fungal and anti bacterial agents.

According to a fifth aspect of the present invention there is provided a method of screening a first and a second compound, to test whether or not said compounds has efficacy for use in combination as a chemotherapy, comprising:
(i) exposing said compounds to Topoisomerase II and evaluating whether or not said compounds bind thereto;
(ii) exposing said compounds to Heat Shock Protein 90 and evaluating whether or not said compounds bind thereto; and
(iii) selecting a first and second compound, wherein at least one compound binds to Topoisomerase II and at least one compound binds to Heat Shock Protein 90 for use in combination as a chemotherapy.

It will be appreciated that the method according to the fifth aspect of the invention may be adapted such that it is used to test whether or not a single compound may have a novel use in chemotherapy. Therefore according to a sixth aspect of the invention there is provided a method of screening a compound, to test whether or not said compound has efficacy for use in chemotherapy, comprising exposing said compound to Topoisomerase II and Heat Shock Protein 90 to evaluate whether or not said compound prevents interaction between Topoisomerase II and Heatshock Protein 90.

Compounds screened according to the fifth or sixth aspects of the invention represent candidate chemotherapeutic agents. The screening methods are is based upon the inventors realisation that interaction between Topoisomerase II and Heat Shock Protein 90 is closely related to undesirable cell growth (carcinogenesis and the like). It will be appreciated that the pharmaceutical industry will be able to use the methods according to the fifth or sixth aspect of the invention to identify candidate medicaments for further investigation as anti-cancer agents.

A preferred technique for carrying out the methods of the fifth and sixth aspects of the invention is to exposure the compounds to be tested to Topoisomerase II and Heat Shock Protein 90 used as binding partners in an interaction trap. Many forms of interaction trap are known to the art. Preferably a yeast two-hybrid interaction trap is employed. Yeast two-hybrid screening is a strategy for screening for interaction between proteins. Yeast two-hybrid screening used according to the invention may involve expression of translational fusions of (a) Topoisomerase II and part of a reporter gene; and (b) Heatshock Protein 90 fused in-frame with the other part of the reporter gene. When the fusion proteins are expressed, interaction between (a) and (b) allows the reporter to assemble and generate a signal. Test compounds that represent candidate chemotherapeutic agents prevent interaction between (a) and (b) and may be identified because no reporter signal is produced from samples containing the candidate.

It will be appreciated that any other form of interaction trap may be used to put the invention into practice. Suitable examples included techniques such as mammalian two-hybrid, bacterial two-hybrid or alternatively various types of pull down assay.

When the methods relate to the disruption of protein-protein interactions based on the yeast two-hybrid technique it is preferred that yeast are used that are permeable to the tested compounds. Examples of drug permeable yeast which may be used according to the invention include MDS or ISE 2 mutations (e.g. strains carrying these mutations (ISE2), JJ700, BJ201). Suitable strains are disclosed in Hammonds et al. Antimicrob Agents Chemother. April 1998;42(4):889-94.

It will be appreciated that the methods according to the fifth or sixth aspects of the invention may be adapted to identify compounds that promote interaction between Topoisomerase II and Heatshock Protein 90 (rather than inhibit such interaction) Such an adapted test represents a good method for evaluating whether or not a test compound is likely to be carcinogenic. Therefore according to a seventh aspect of the present invention there is provided a method of screening a compound, to test whether or not said compound is carcinogenic, comprising exposing said compound to Topoisomerase II and Heatshock Protein 90 to evaluate whether or not said compound promotes interaction between Topoisomerase II and Heatshock Protein 90.

Accordingly any compound, identified according to the seventh aspect of the invention, that promotes interaction between Topoisomerase II and Heatshock Protein 90 is likely to be carcinogenic. The method may be used to screen compounds to assess whether or not they are safe to be used by the public. For instance cosmetics, foodstuffs, candidate therapeutic agents etc may all be tested to investigate whether or not they may cause cancer. The method according to the seventh aspect of the invention may also be used for environmental monitoring. For instance, the test may be used to evaluate whether or not effluent from a factory may contain carcinogenic compounds.

The discovery that Topo II and Hsp90 interact has further enabled the inventor to develop a test whereby the measurement of HSP90 and Topoisomerase II protein levels in cells is used as a diagnostic aid. According to an eighth aspect of the present invention there is provided an in vitro method for diagnosing whether or not a subject has, or is likely to develop cancer, comprising:
(i) detecting the level of activity or expression levels of HSP90 and Topoisomerase II from a sample of cells from said subject; and
(ii) comparing the level of activity or expression levels of HSP90 and Topoisomerase II in said sample relative to activity expression levels of HSP90 and Topoisomerase II from a non-cancerous sample.

The method according to the eighth aspect of the invention indicates that a subject is at risk of developing cancer if the activity or expression levels of Topo II or HSP90 are raised relative to control values (e.g samples from an individual without cancer or from non-cancerous tissues from the subject).

Preferably a first sample is taken from a tissue which is suspected to be cancerous and a second sample is taken from normal tissue (i.e non-cancerous tissue) from the same subject.

The method according to the eighth aspect of the invention may be adapted for determining the sensitivity of a subject to a specific combination of first and second agents according to the invention (i.e. an HSP90 inhibitor and a Topoisomerase II inhibitor). Thus according to a ninth aspect of the present invention there is provided an in vitro method for evaluating the suitability of chemotherapeutic treatment for administration to a subject, comprising:
(i) detecting the level of activity or expression levels of HSP90 and Topoisomerase II from a sample of cells from said subject; and
(ii) comparing the level of activity or expression levels of HSP90 and Topoisomerase II in said sample relative to activity expression levels of HSP90 and Topoisomerase II from a non-cancerous sample.

According to a tenth aspect of the present invention there is provided an in vitro method for monitoring the effectiveness of a chemotherapy for treating a subject, comprising:
(i) detecting the level of activity or expression levels of HSP90 and Topoisomerase II from a sample of cells from said subject; and
(ii) comparing the level of activity or expression levels of HSP90 and Topoisomerase II in said sample relative to activity expression levels of HSP90 and Topoisomerase II from a non-cancerous sample.

The methods of the eighth, ninth or tenth aspects of the invention may be based on an immuno-technique to detect the level of HSP90 and Topoisomerase II in a sample. A Western blot technique, for instance as described in Example 1, may be employed. Alternatively transcript levels of HSP90 and Topoisomerase II may be detected in a sample. To this end real time PCR may be employed with primers based on the known nucleotide sequence of HSP90 and Topoisomerase II.

The invention will be further illustrated in the non-limiting Example and figures, in which:

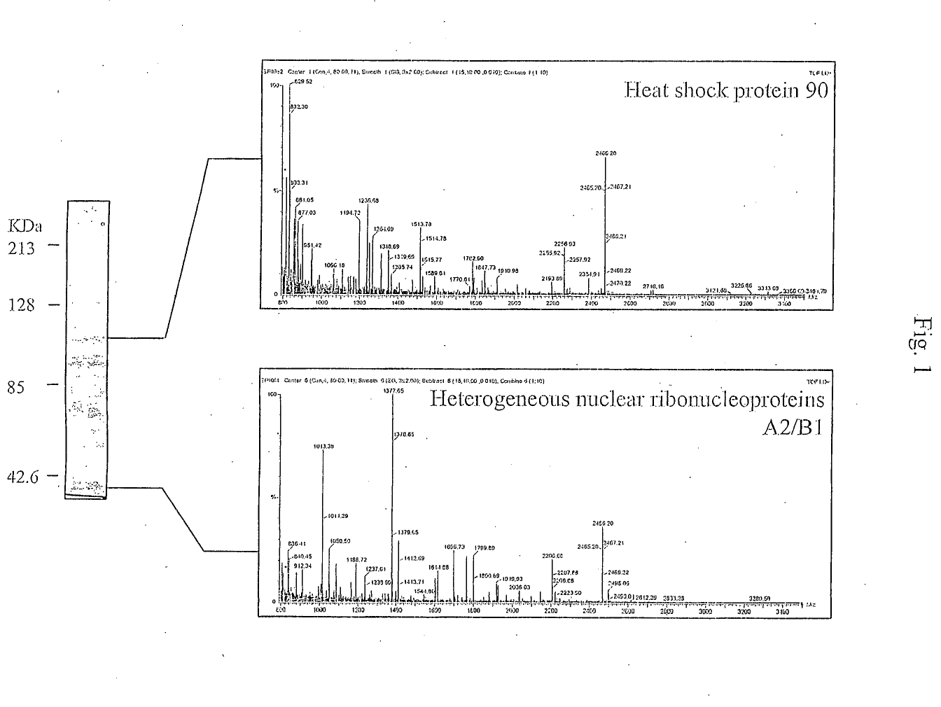

FIG. 1 illustrates mass fingerprinting data which identified that HSP 90 β was associated with native topoisomerase IIα following immunoprecipitation in Example 1;

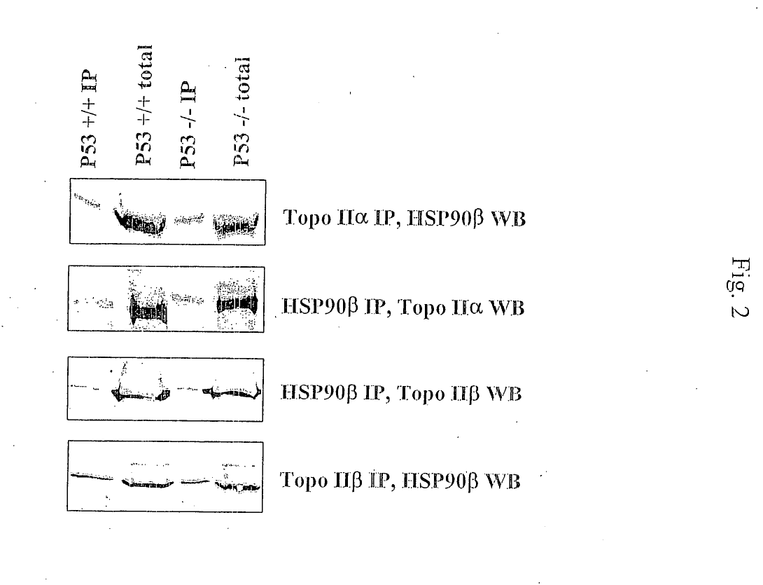

FIG. 2 shows western blots of counter immunoprecipitations and probing of the blots, demonstrating that HSP 90 β and topoisomerase IIα come down in pull down experiments in Example 1;

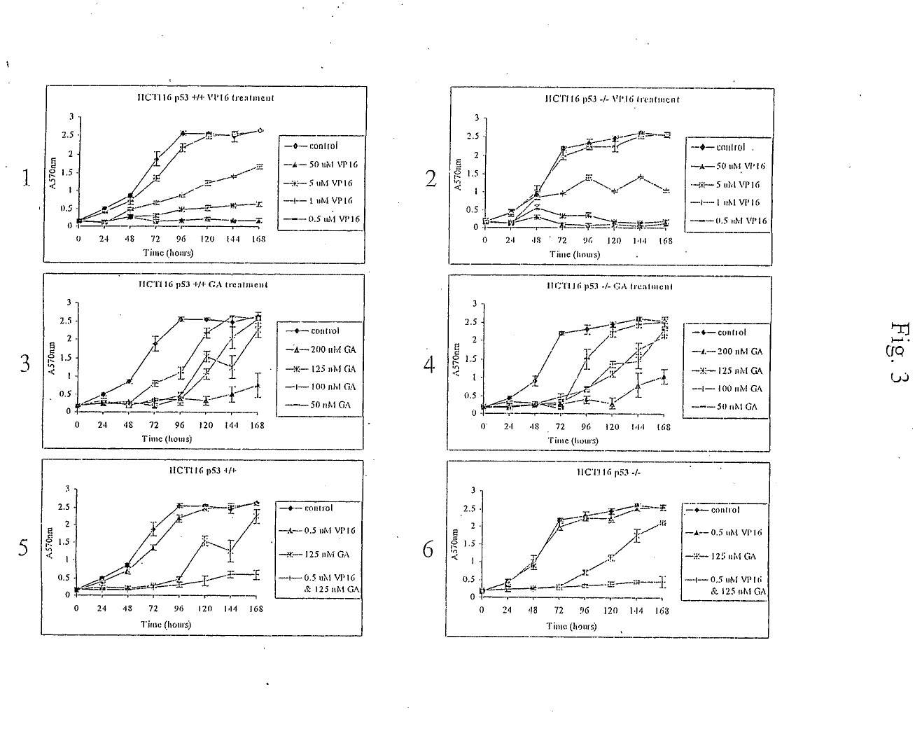

FIG. 3 illustrates the effect of inhibitors of HSP 90 and inhibitors of topoisomerase II on proliferation and cell killing in cell lines in Example 2;

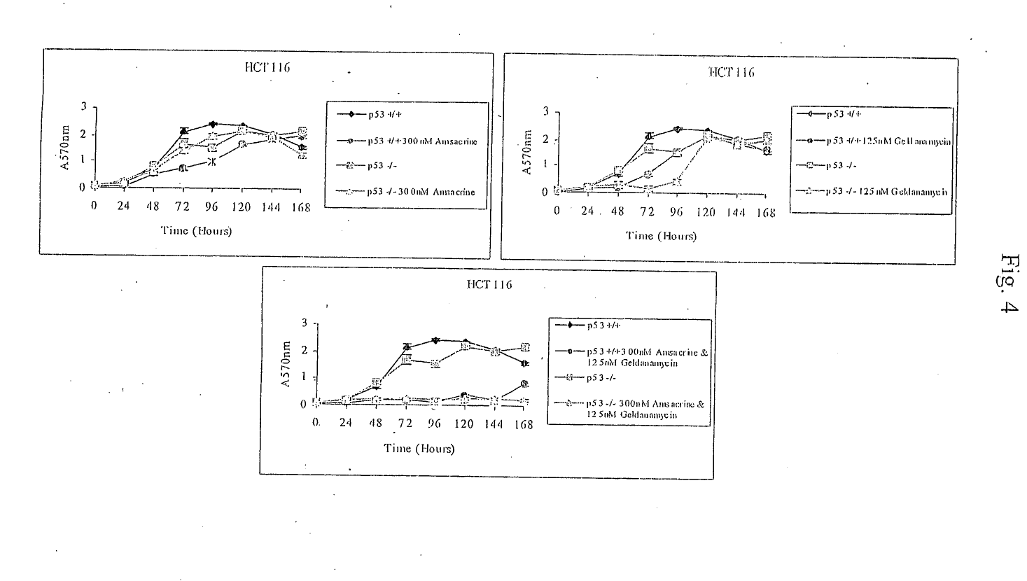

FIG. 4 illustrates the effect of 300 nM Amsacrine and 125 nM Geldanamycin on proliferation and cell killing in cell lines in Example 2;

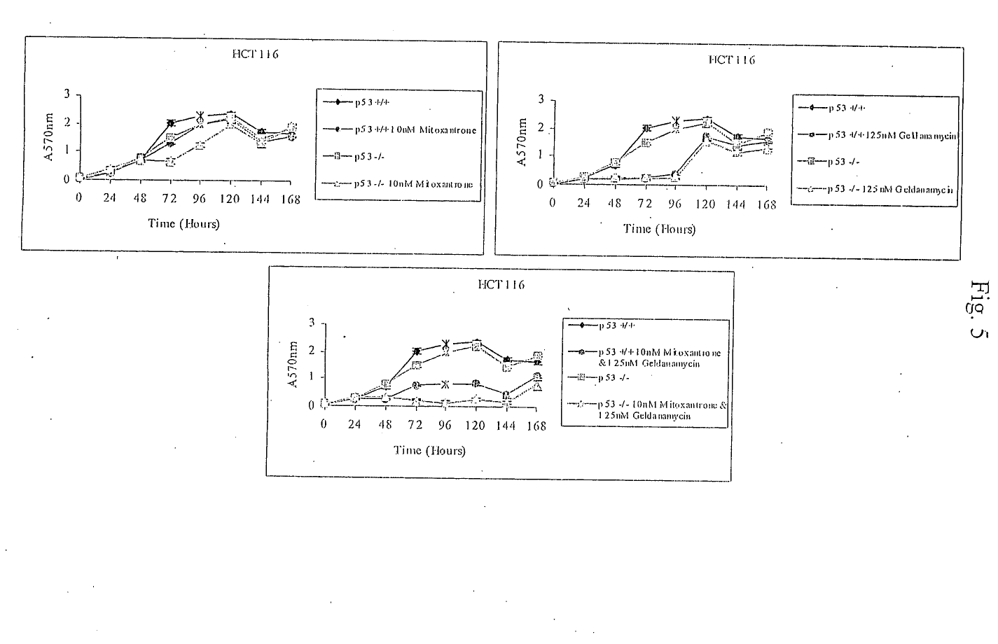

FIG. 5 illustrates the effect of 10 nM Mitoxantrone and 125 nM Geldanamycin on proliferation and cell killing in cell lines in Example 2;

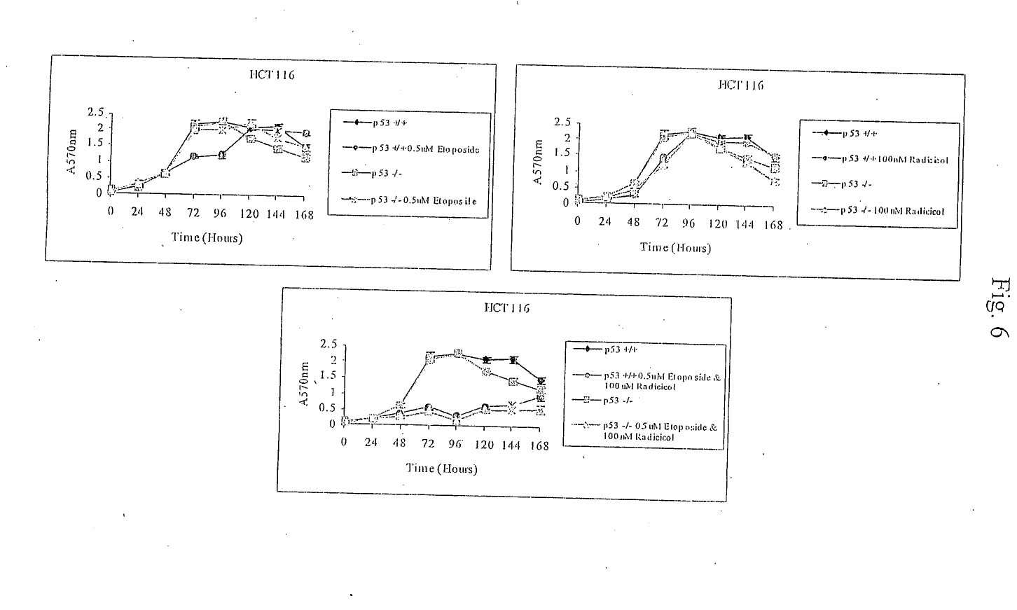

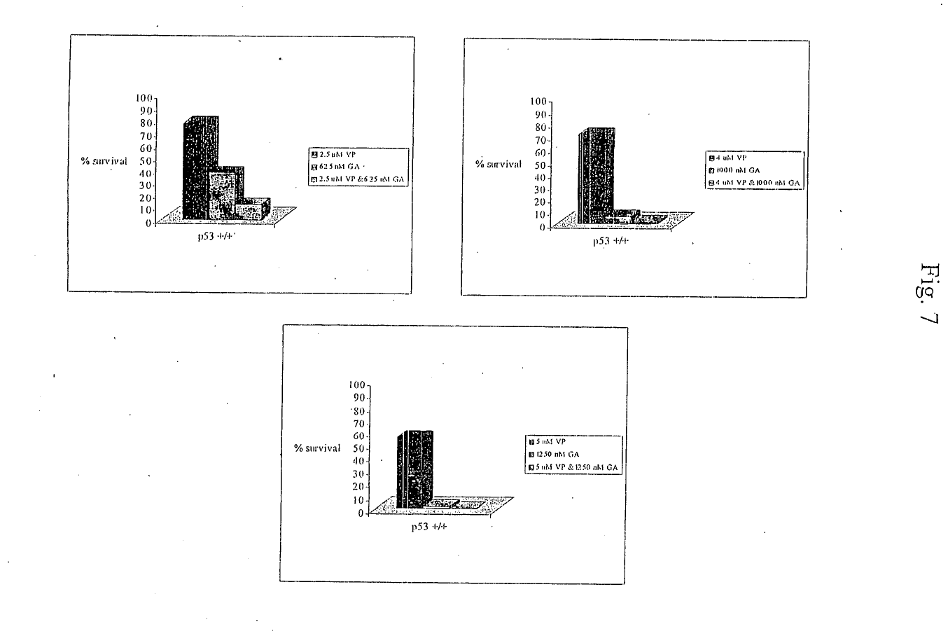

FIG. 6 illustrates the effect of 0.5 μM Etoposide and 100 nM Radicicol on proliferation and cell killing in cell lines in Example 2;

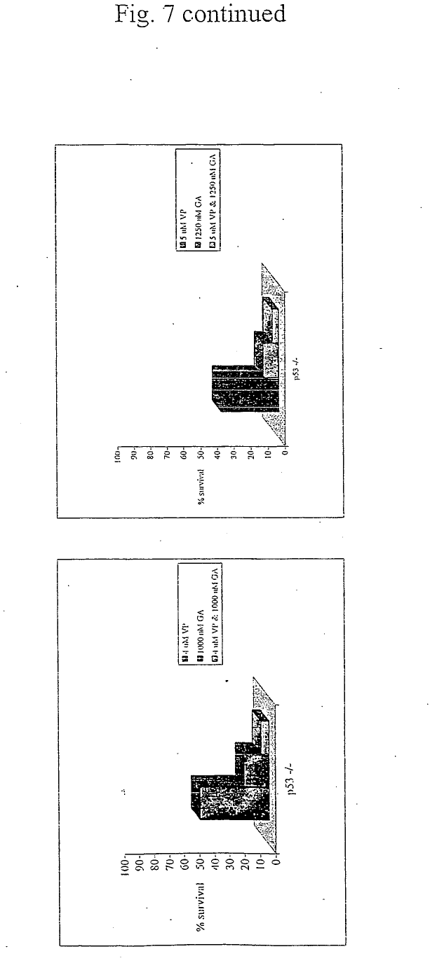

FIG. 7 illustrates data from a clonogenic assay for cell killing (1 hr exposure) for the drugs specified in the legends in Example 2;

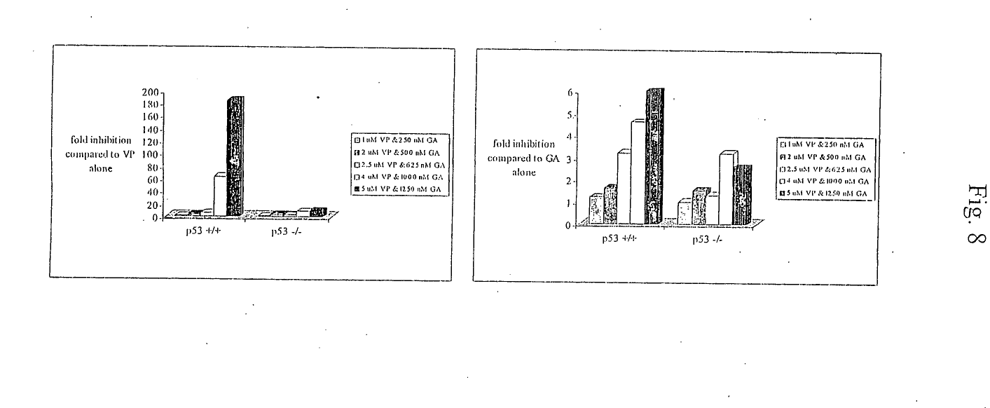

FIG. 8 demonstrates the relative fold change to single drug treatment for the data presented in FIG. 7.

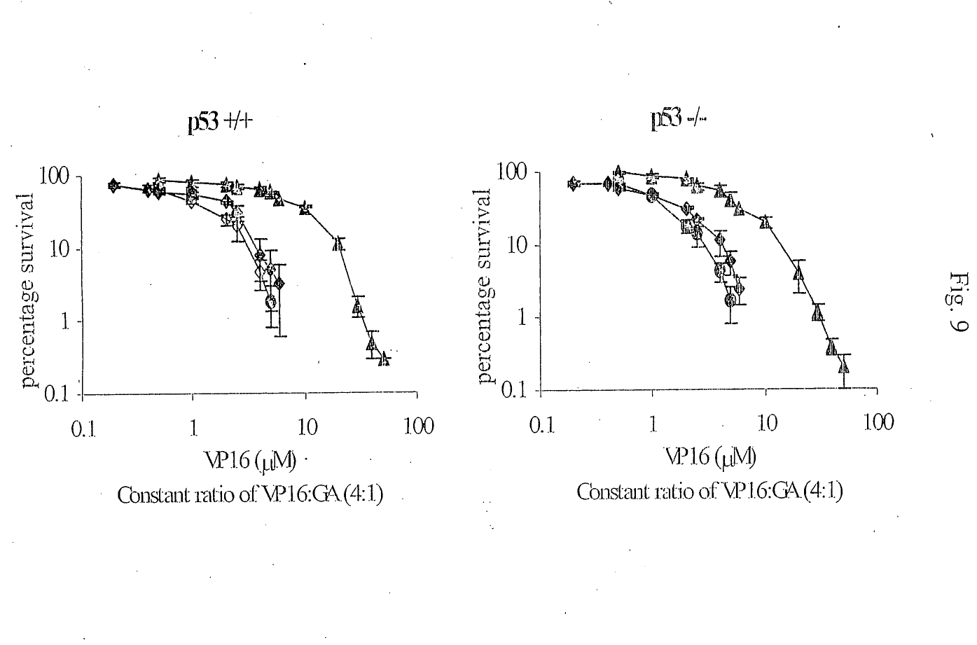

FIG. 9 illustrates the effect of drug combinations on HCT116 clonogenic survival in p53 +ve and p53 −ve cells for VP16 (▴), GA (♦) and VP16 & GA combinations (●);

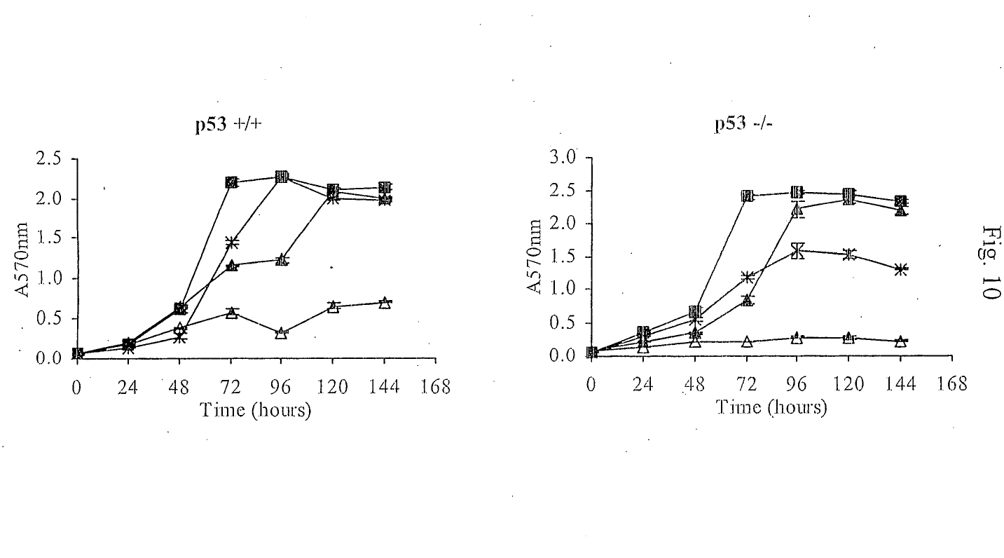

FIG. 10 illustrates Growth inhibition assays for HCT116 cell lines (SRB) Etoposide and Radicicol for Control (▪), 0.5 μM VP16 (▴),100 nM RC ( [symbol] ), 0.5 μM VP16 & 100 nM RC combination (A) and each point represents the mean of six replicates and the error bars represent standard error in Example 2;

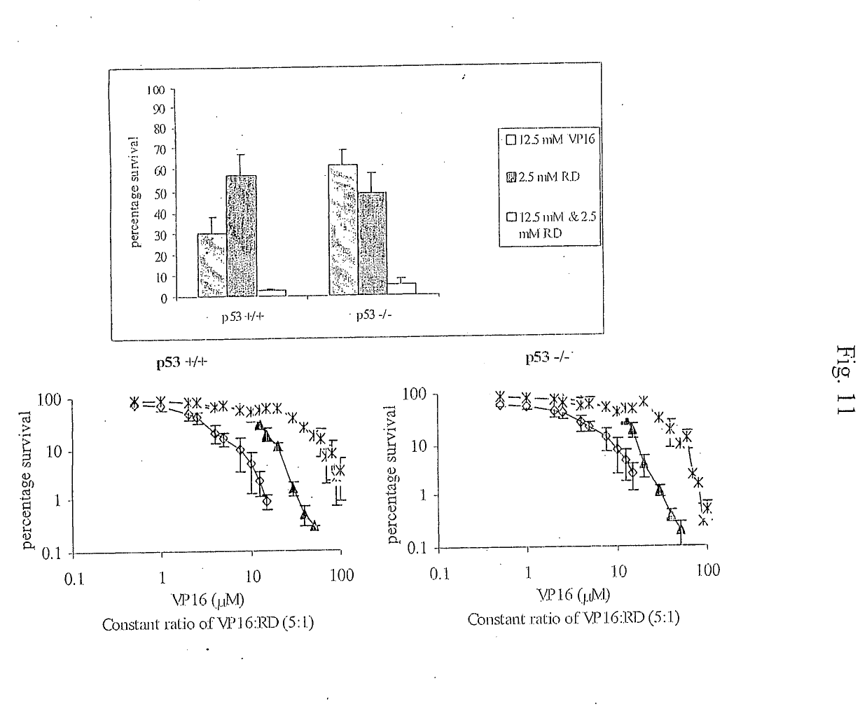

FIG. 11 illustrates the effect of drug combinations on HCT116 clonogenic survival for Etoposide (VP16) and radicicol for VP16 (▴), RC ( [symbol] )and VP16 & RC combination (◯)in Example 2;

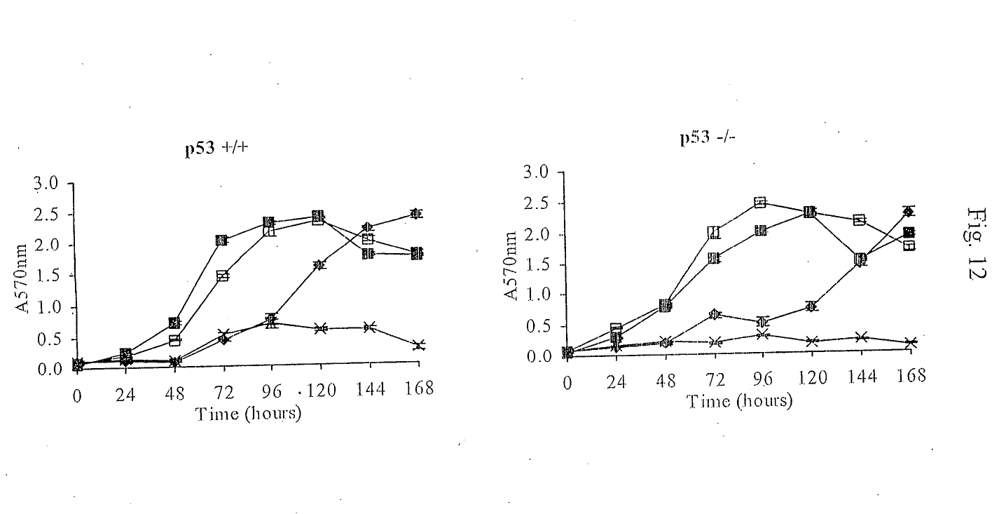

FIG. 12 illustrates Growth inhibition assays for HCT116 cell lines (SRB) Mitoxantrone and Geldanamycin for Control (▪), 10 nM MX (□), 125 nM GA (♦) and 10 nM MX & 125 nM GA combination (X) in Example 2;

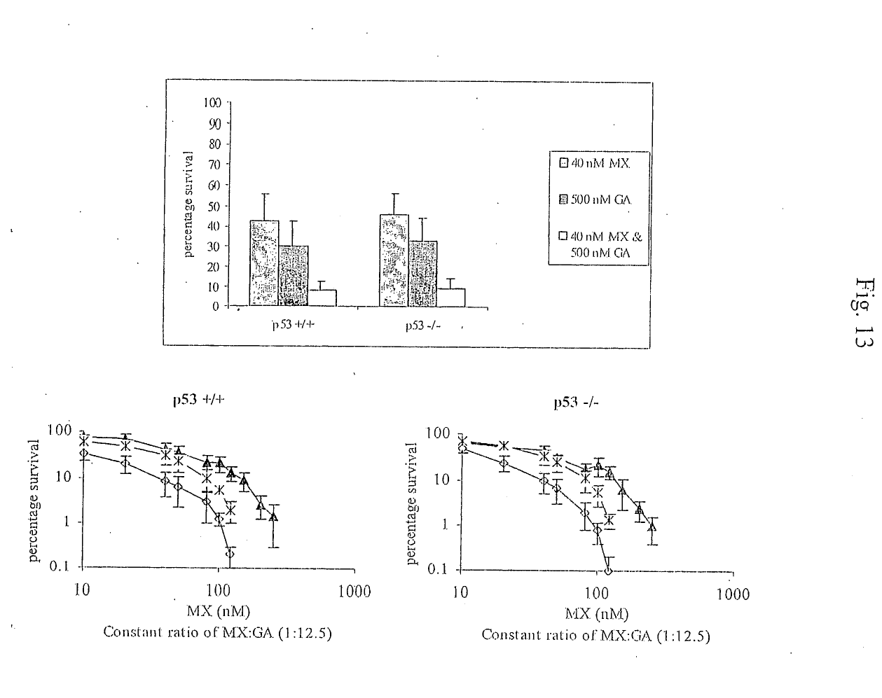

FIG. 13 illustrates the effect of drug combinations on HCT116 clonogenic survival Mitoxantrone and geldanamycin(GA) for MX (▴), GA ( [symbol] ) and MX & GA combination (◯) in Example 2;

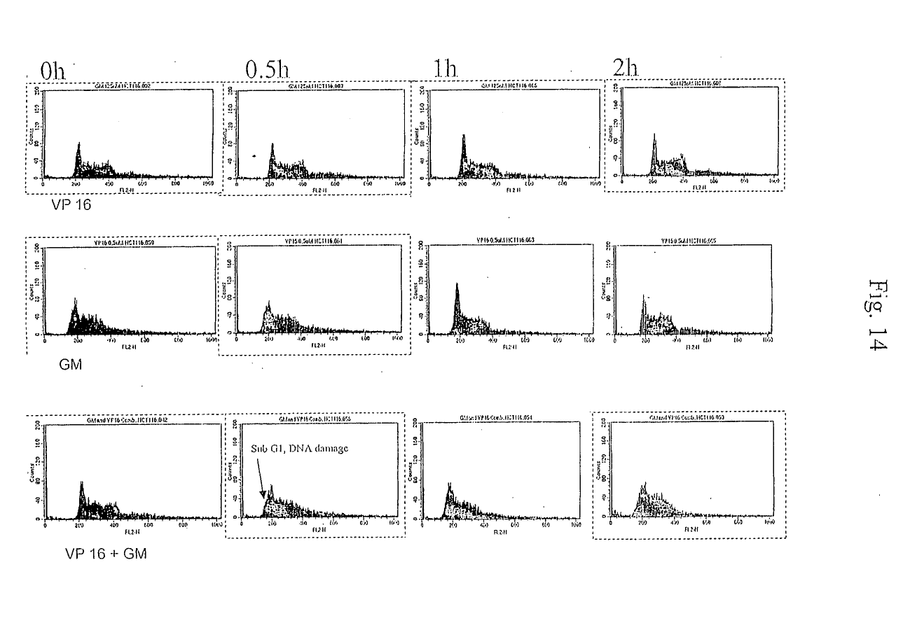

FIG. 14 illustrates the effect of inhibitors of HSP 90 and inhibitors of topoisomerase II on DNA damage after 30 minutes treatment in Example 2;

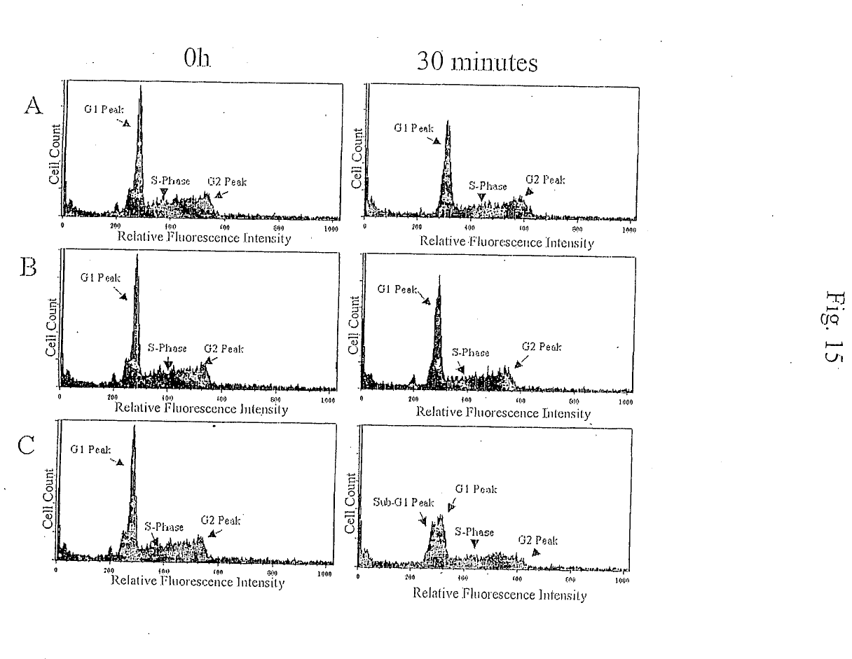

FIG. 15 illustrates the effect of inhibitors of HSP 90 and inhibitors of topoisomerase II on DNA damage in the leukaemic cell line K562 after 30 minutes treatment in Example 2;

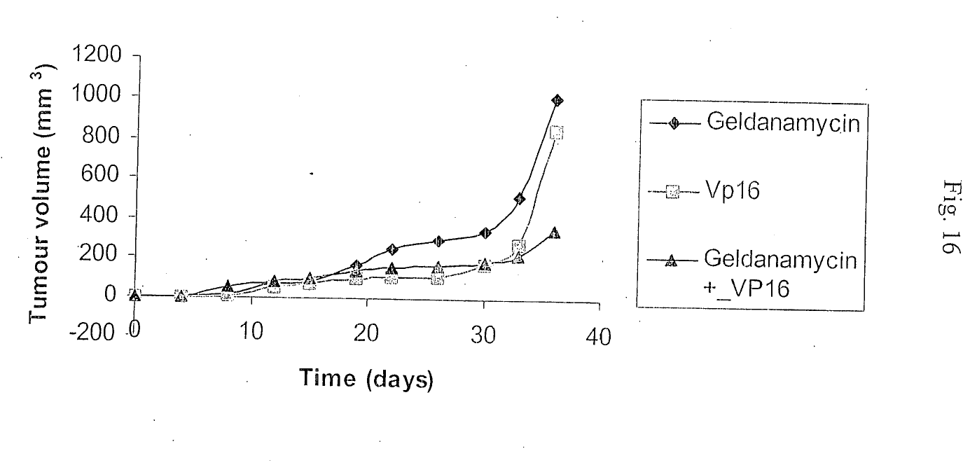

FIG. 16 is a graph illustrating a xenograft regression study as referred to in Example 3;

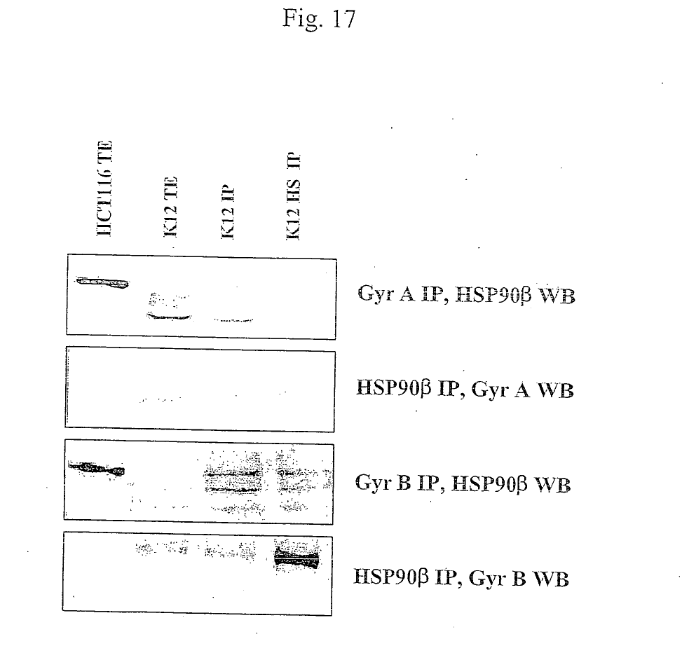

FIG. 17 shows western blots of counter immunoprecipitations and probing of the blots, demonstrating that HtpG (Hsp90) and DNA gyrase (topoisomerase II) come down in pull down experiments performed in bacterial samples in Example 4

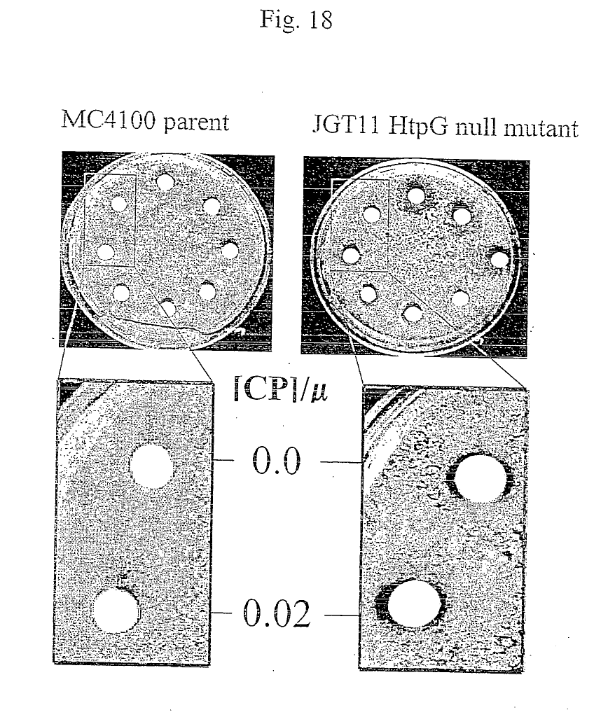

FIG. 18 shows inhibition of bacterial growth caused by treating an HtpG knock-out E. coli strain and a wild type strain with Ciprofloxacin (a topo II inhibitor) in Example 5; and

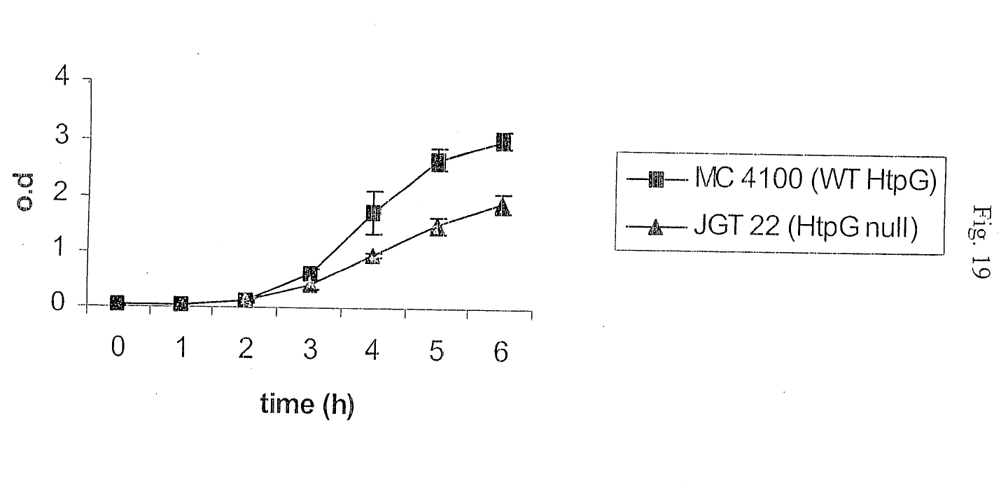

FIG. 19 illustrates culture growth was reduced for a htpG null bacteria mutant treated with Ciprofloxacin to a greater extent than in wild type bacteria treated with Ciprofloxacin in Example 5.

### EXAMPLE 1

Experiments were conducted that established HSP90 and Topoisomerase II interact and influence cell growth. This discovery lead the inventor to develop the various aspects of the invention described herein.

Methods

Immunoprecipitations

100 mm dishes were seeded with 3×10⁶ cells and allowed to adhere overnight. Media was placed with fresh media alone (control) or containing e.g. 50 μM VP16 for 24 hours. Cells were wash twice with wash buffer (0.4 mM EDTA, 10 mM sodium fluoride, 10 mM sodium pyrophosphate, 0.4 mM sodium orthovanadate) and incubated on ice with 250 μl cell lysis buffer (50 mM Tris HCl pH 8.0, 425 mM NaCl, 1 mM EDTA, 10 mM sodium fluoride, 0.5 mM sodium orthovanadate, 1% v/v igepal CA-630, 5% w/v deoxycholic acid, 0.1% w/v SDS) containing protease inhibitor cocktail III (Calbiochem). Cells were scraped on ice, sonicated for 30 seconds and cell debris removed by centrifugation at 14,000×g for 30 minutes at 4° C. Cell lysates were then pre-cleared by incubation with 25 μl of 10% w/v protein A sepharose CL-4B (Amersham Pharmacia Biotech) in PBS for 1 hour rotating at 4° C. Samples were spun briefly at maximum speed in a 4° C. benchtop centrifuge and supernatants removed to fresh microfuge tubes. 5 μg of either anti-topoisomerase IIα (Labvision) or anti-heat shock protein 90β (Labvision) antibodies were added to cell lysates and incubated at 4° C. overnight. 50 μl of 10% w/v protein A sepharose in PBS was added and samples allowed to precipitate by rotating at 4° C. for 1 hour. Samples were spun briefly at maximum speed in a 4° C. benchtop centrifuge and supernatants discarded. Immunoprecipitates were washed with 250 μl cell lysis buffer, resuspended in 60 μl IPG buffer (7 M urea, 2 M thiourea, 4% w/v CHAPS, 40 mM Tris base, 1% w/v DTT) and analysed by one-dimensional (1-D) electrophoresis.

1-D Electrophoresis and Immunoblotting

Total protein extracts and immunoprecipitations were separated by 7.5% or 12% SDS-PAGE under reducing conditions. Gels were then either stained using Colloidal blue concentrate (Sigma) in 20% v/v methanol or blotted onto nitrocellulose membrane. Blots were probed with either rabbit primary antibodies against human Topoisomerase IIα or Heat Shock Protein 90β, or mouse primary antibodies against human heat shock protein 70 (Labvision). Anti-rabbit and anti-mouse IgG secondary antibodies conjugated with horseradish peroxidase (DAKO) were detected by Supersignal West Dura Extended Substrate (Pierce) and imaged using a Fluor-S bioimager (BioRad).

In-Gel Digestions and Mass Spectrometry

Bands were excised from gels, placed into 0.5 ml microfuge tubes pre-washed with acetonitrile followed by methanol. Gel slices were washed with 100 ml 50% v/v acetonitrile, 25 mM ammonium bicarbonate for 15 minutes with occasional gentle agitation. Samples were briefly spun and supernatant discarded. Gel slices were then dried in a SpeedVac for approximately 30 minutes and rehydrated overnight at 37° C. in 4 μl of 10 ng/μl trypsin (Promega) in ammonium bicarbonate. 4 μl of d H₂O was added to each sample and allowed to soak for 15 minutes. Peptides were then extracted by the addition of 7 μl of 30% v/v acetonitrile, 0.1% v/v trifluoroacetic acid (TFA) and brief vortexing. 0.5 μl of sample was placed onto a 96 well target and mixed with 0.5 μl of 10 mg/ml α-cyano-4-hydroxycinnamic acid (HCCA; dissolved in 495 μl acetonitrile, 495 μl ethanol, 10 μl 0.1% TFA) containing 0.5 fmol ACTH as an internal standard. Matrix assisted laser desorption ionisation time of flight (MALDI-TOF) mass spectrometry was performed by Dr. Stephen Pennington, Dept. Human Anatomy and Cell Biology, using a Micromass MALDI. Spectra obtained were analysed using MassLynx and ProteinProbe software programmes and compared to theoretical tryptic digests created from a FASTA database.

Results

Protein-Protein Interactions

Protein association studies were conducted using 1 dimensional SDS-PAGE analyses of co-immunoprecipitated proteins. Immunoprecipitation were undertaken with commercial antibodies against the native protein, and binding partners were identified by 1 dimensional SDS-PAGE and peptide mass fingerprinting by MALDI-TOF mass spectroscopy and western blot analysis.

The mass fingerprinting data identified HSP90 β as being associated with native topoisomerase IIα following immunoprecipitation, see FIG. 1. In FIG. 1, HCT 116 cells were exposed to 50 μg/ml etoposide for 24 hr. Proteins were isolated by immunoprecipitation with anti-topoisomerase IIα antibodies. Peptide mass fingerprints were obtained by a MALDI mass spectrometer (M@LDI, Micromass).

The counter precipitation was performed (IP with antibodies against HSP90β) and topoisomerase II was demonstrated to be associated with HSP90. FIG. 2 shows western blots of counter immunoprecipitations and probing of the blots, demonstrating that the corresponding proteins come down in pull down experiments.

The results, MALDI and IP, demonstrate that there is a physical interaction between Topoisomerase II and HSP 90.

Drug Target

The inventor realised that the interaction between these two proteins represents a new drug target and went on to assess the effect of modulators of these proteins in combination for chemotherapy (see Example 2)

### EXAMPLE 2

Example 1 illustrates there was a physical interaction between Topo II and HSP90. We therefore tested the effect of combining drugs that had a specific effect on Topoisomerase II and a specific effect on HSP90. We tested at least two classes of drug that inhibit HSP90 and at least four that inhibit topoisomerase II. The combination of an HSP90 inhibitor and a topoisomerase II inhibitor show a synergistic effect (see below).

Methods

Established Cell Culture

The isogenic p53 human colon cancer cell line (WT and KO), HCT116, was a kind gift from Bert Vogelstein. Cells were maintained in McCoys 5A medium (Sigma) supplemented with 10% foetal calf serum (Gibco) at 37° C. in a 5% CO₂ enriched humidified environment, Penicillin and Streptomycin.

Standard Cell Lines as Above Except:

K562 RMPI 1640 (Sigma), SK-MEL-3 McCoys (Sigma), OAW42 DMEM supplemented with 1 mM sodium pyruvate 10 μg/ml insulin & NCI-H125 RPMI 1640 (Sigma), HT29 DMEM (Sigma).

Growth Inhibition Assay

96 well flat-bottomed plates were seeded with 3×10³ cells per well and allowed to adhere overnight. Media was then replaced with fresh media alone (control) or containing test drugs eg 0.1 to 50 μM etoposide (VP16), 50 to 200 nM geldanamycin (GA) and combinations of both. At fixed time points, cells were fixed with 3:1 methanol:acetic acid and stained with 0.4% w/v sulforhodamine B (Sigma) in 1% v/v acetic acid for 30 minutes. Plates were then washed twice with 1% v/v acetic acid, the dye solubilised with 100 μl per well of 10 mM Tris pH 10.4 and read at A570 nm using a Benchmark microplate reader (BioRad).

Clonogenic Assay

Cells were plated at a density of 1000 cells per well in 6 well plates and allowed to adhere overnight. Cells were treated with e.g. 0.5 to 50 μM VP16, 50 to 1500 nM GA or combinations of the two for 1 hour. Cells were then washed twice with PBS and re-incubated with fresh medium for 10 days. Media was then removed and cells were fixed with 70% v/v methanol for 1 minute. Cells were then stained with 0.2% w/v crystal violet in 70% v/v ethanol for 10 seconds, washed with dH₂O and allowed to air dry. The number of colonies formed of>50 cells each were counted.

Drugs were used in the following concentrations for growth inhibition and clonagenic assays:
Geldanamycin 1-1500 nM
Radicicol 5 nM-37.5 μM
Etoposide 0.01-250 μM
Merbarone 0.01-100 μM
Amsacrine 0.01 -200 μM
ICRF 159 0.01 μM-2 mM
Mitoxantrone 0.01 nM-100 μM
   Flow Cytometry Protocol for Cell Cycle Analysis.

1. Seed cells eg HCT116+/+ or K562 cells in small petri dish or 6 well plate using 5 ml of 1×10⁶ cells/ml in appropriate medium. For HCT116+/+ cell line use McCoys 5A Medium supplemented with 10% Foetal Calf Serum (FCS) and Penicillin and Streptomycin. For K562 cell line use RPMI 1640 Medium supplemented with 10% FCS and Penicillin and Streptomycin.

2. Leave to attach overnight for adherent cell lines in incubator at 37° C. 5% CO₂ atmosphere.*
* For suspension cell lines, spin cells down and resuspend at between 2-4×10⁵ cells/ml in medium supplemented with the drug treatment required.

3. Dose with 5 ml of drug/control for required time course in incubator at 37° C. 5% CO₂ atmosphere. 125 nM Geldanamycin, 0.5 μM VP16, or 125 nM Geldanamycin and 0.5 μM VP16 combination.

4. After treatment, remove medium from well to a universal tube.**
* * For suspension cell lines ignore steps 6 and 7.

5. Wash well with 500 μl PBS and remove to same universal.

6. Add 500 μl trypsin and wait for detachment.

7. Add trypsin and cells to universal and rinse out the well with some of the medium from the universal.

8. Spin cells at 4° C. at 2500 rpm for 5 mins.

9. Remove supernatant and resuspend pellet in 500 μl PBS

10. Transfer to Falcon tube and spin at 4° C. at 2500 rpm for 5 minutes

11. Remove supernatant and add 500 μl ice-cold 70% ethanol, and leave in fridge for 2-5 minutes.

12. Spin cells at 4° C. at 2500 rpm for 5 mins.

13. Wash twice in 1 ml PBS.

14. Add 40 μl of 100 μg/ml ribonuclease A for 5 mins at room temperature.

15. Add 400 μl of 50 μg/ml propidium iodide (Sigma) and incubate for 15 minutes.

16. Analyse on FACSVantage SE (Becton Dickinson) using 488 nm laser for excitation, and collecting fluorescence above 585 nm (FL-2). Collect data using CellQuest Pro v4.0. Analyse data using Mod Fit LT v3.0

Results

This is the first time that disruption of two interacting proteins has been specifically demonstrated as a chemotherapeutic treatment (Rational chemotherapy). This was tested using an inhibitor of HSP90 and an inhibitor of Topoisomerase II in combination; the beneficial effect of disrupting the interaction between the two proteins is shown in FIG. 3, for proliferation. In FIG. 3:
1 & 3 represent single drug treatment on WT p53 cells;
2 & 4 represent single drug treatment on p53 KO cells; and
5 & 6 represent combination treatment showing only inhibition of proliferation with the combination of drugs.

FIG. 4 illustrates the effect of 300 nM Amsacrine and 125 nM Geldanamycin on proliferation and cell killing in HTC 116 cells.

FIG. 5 illustrates the effect of 10 nM Mitoxantrone and 125 nM Geldanamycin on proliferation and cell killing in in HTC 116 cells.

FIG. 6 illustrates the effect of 0.5 μM Etoposide and 100 nM Radicicol on proliferation and cell killing in HTC 116 cells.

The clonogenic assay for cell killing (1 hr exposure) is shown in FIG. 7 for the drugs specified in the legends. FIG. 8 demonstrates the relative fold change to single drug treatment. The time required for the killing process is an exposure of 1 hour or less as demonstrated by the clonogenic assay.

Data for specific drug combinations in proliferation and clonogenic assays are illustrated in FIG. 9-13.

DNA damage was looked at during early time points. FIG. 14 demonstrates that there is a population of DNA that is sub G1 at 30 minutes. This is was also found to be the case in another cell line (leukaemic, K562) as illustrated in FIG. 15. In this figure A: 125 nM Geldanamycin; B: 0.5 μM VP16; and C: 125 nM Geldanamycin and 0.5 μM VP16

For FIG. 15 the staining Method involved fixing Cells in 70% Ethanol for 5 minutes, then treated with RNase A for 5 minutes. Stained with 50 μg/ml Propidium Iodide, a fluorescent dye which intercalates stoichiometrically with DNA.

The presence of the Sub-G1 Peak after 30 minutes of treatment C in FIG. 15 indicates that DNA damage has occurred.

Isobolar Relations

The isobolar relations were calculated to quantify the synergistic combination of the two agents used according to the invention.

The isobalar relation is calculated in the light of the fact that two drugs used in combination may produce enhanced or reduced effects. The degree of enhancement or reduction is measured from the interaction index (γ), defined by the isobolar relation, which indicates the changed potency of the combination.
(a/A)+(b/B)=γ
Where:A=drug A alone; B=drug B alone; and a, b=combination dose to produce desired effect
If γ1=additive; <1=super-additive (synergistic); and >1=sub-additive

The isobolar relations for the specified combination of drugs in HCT116 cells were as follows:

SRB Assay: (for Both Wild Type and p53 Knockouts)

| | |
|---|---|
| γ VP & GA = | 0.62 |
| γ VP & 17-AAG = | 0.62 |
| γ VP & RD = | 0.45 |
| γ MX & GA = | 0.62 |

Clonogenic Assay:

| | |
|---|---|
| γ VP & GA = | 0.92 (both WT & p53 KO) |
| γ VP16 & RD = | 0.50 (WT) |
| γ VP16 & RD = | 0.81 (KO) |
| γ MX & GA = | 0.97 (WT) |
| γ MX & GA = | 0.90 (KO) |

It will therefore be appreciated that the combination of the two agents produces a synergistic effect (i.e. cell killing at concentrations, where there is little or no effect with single drugs. At least 3-5 times greater than the drug used in isolation). The action is independent of p53 status.

### EXAMPLE 3

The effect of the combinatin therapy according to the invention was assessed in an animal model of tumour development. The data illustrated that agents used according to the invention reduce tumour size in vivo.

Xenograft Regression Assay

Male CD1 Nude mice aged 10 weeks and weighing approximately 25 g, were used. The mice had free access to food and water, under an alternating 12 hr light/dark regime. Mice were injected subcutaneously in each flank with a suspension containing 1×10⁶ HCT116 cells.

Animals were examined for tumour growth on a daily basis. Tumour volume was examined for evidence of growth on a daily basis. Tumour volume was estimated as the product of three perpendicular measurements made using analogue calipers (ie approximated to a cube).

Treatment with test agents was initiated when the mean individual tumour volume exceeded 1,000 mm³. The mice were dosed at day 11 and day 22—(Geldanamycin 10 mg/kg, VePesid 5 mg/kg, MTD Geldanamycin 22 mg/kg (a non toxic form of 17AAG which can be tolerated at much higher doses) MTD VePesid 70 mg/kg.

Tumour dimensions were subsequently measured every third day. In accordance with the Animals (Scientific Procedures) Act of 1986 the experiments were halted when the individual tumour volume exceeded 1000 mm³.

FIG. 16 illustrates the Xenograft data and demonstrates that a combination of drugs according to the invention causes a significant reduction in tumour size.

### EXAMPLE 4

The Experiments conducted in Example 1 on HCT116 cells were repeated in the bacterial E. coli strain K12. To demonstrate that HSP90 and Topoisomerase II interact and influence microbial cell growth and susceptibility to chemotherapy.

The experimental procedures outlined in Example 1 were repeated with the exception that known antibodies raised against the bacterial equivalent of Topoisomerase II (DNA gyrase, Gyr A and Gyr B respectively) were used in co-immunoprecipitation experiments. As in example 1 the antibody against the human Hsp90 (which recognised the bacterial equivelant, HtpG) was used for this series of experiments.

FIG. 17 shows western blots of immunoprecipitations and counter immunoprecipitations probed with the complementary antibodies, demonstrating that the corresponding proteins come down in pull down experiments and thereby illustrating that Hsp90 and Topoisomerase II homologues in bacteria interact and influence cell growth and death.

### EXAMPLE 5

Geldanamycin inhibits the activity of HtpG in vitro. However, to demonstrate the effect of a combination therapy on bacteria E.coli htpG null mutants were used. This also demonstrates that knocking out one of the target genes genetically has as similar effect to using a drug.

Agar Sensitivity Plates:

Methods

To demonstrate the effect of various levels of HtpG (bacterial Hsp90) expression we used E.coli strains that were htpG null, the parental strain MC4100 and a strain that over expresses HtpG (plasmid expressing HtpG).

These strains were used in plate growth assays:
HtpG null: JGT 11 As MC 4100 zba315::kan DhtpG1::lacZ
Parental Strain: MC 4100 araD139 D(argF-lac) U169 rps150 relA1 flbB5301 deoC1
Overexpressing Strain: Top10 F′l⁻ endA1 recA1 hsdR17 (rk⁻mk⁺) supE44 thil gyrA96
(pHtpG pTG10 derived htpG under the control of the native promoter (Chl^{r}))

200 μl of an overnight culture of E.coli strains was spread over the surface of a LB plate. The plates were incubated for three hours at 37° C., and the discs (50 μl of drug) placed onto the plates that were then incubated at 37° C. overnight. Discs containing Quinalone and Ciprofloxacin (CP) (in the range of 0.6-0.01 μM) were used.

The plates were photographed the following day with Fluor-S™ Quantity 1 program and the results are illustrated in FIG. 18.

Results

The agar sensitivity plates were used to assess the sensitivity or resistance of the relevant E.coli strains to Ciprofloxacin (i.e. a topo II inhibitor) or Quinalone (another topo II inhibitor).

The inhibition of bacterial cell growth was assessed by examining the zones of inhibition around each disc containing the topo II inhibitor.

Both Quinalone and CP caused some inhibition of growth in cultures of MC4100 cells. However, increased inhibition was seen in the htpG null mutant. This indicates that combinations of agents according to the invention have a synergistic effect and have a surprising inhibitory effect on microbial growth when used in combination.

FIG. 18 illustrates the effect of CP on growth of MC4100 cells and JGT11. It clearly shows that CP has a significantly greater effect on JGT11 growth, even at concentrations as low as 0.01 μM, whilst the parental strain showed a reduced zone of inhibition at 0.1 μM and no zone of inhibition at 0.025 μM.

Experiments conducted with Top10 (an E. coli strain which over-expresses HtpG) illustrated that the inhibitory effect of a topo II inhibitor was reduced relative to the wild type (MC4100). This demonstrates that the combined inhibition of HSP90 (i.e. HtpG) and topo II is particularly beneficial as a combination therapy.

E.coli Growth Inhibition Assays:

250 μJgt22 and Mc4100 cultures, and 1 ml Top10 culture were added to 50 ml media. 100 μl of 0.01 μM CP was added at the time T=0.

The cultures were incubated at 37° C. during the experiment and the optical density (i.e. number of cells in culture) was measured every hour.

E.coli Strains:

MC 4100 araD139 Δ(argF-lac)U169 rpsL150 relA1 flbB5301 deoC1
JGT 11 As MC 4100 zba315::kan ΔhtpG1::lacZ
JGT 22 As MC 4100 ΔhtpG1::lacZ
Top10 F′λ⁻ endA1 recA1 hsdR17 (rk⁻mk⁺) supE44 thil gyrA96
(pHtpG pTG10 derived htpG under the control of the native promoter (Chl^{r}))

FIG. 19 illustrates that the optical density reading was reduced for htpG null mutants treated with Ciprofloxacin (a Topo II inhibitor) to a greater extent than in wild type bacteria treated with Ciprofloxacin. This demonstrates that the combined inhibition of Topoisomerase II (Topo II) and Heat Shock Protein 90 (HSP90) results in increased bacterial cell death. Accordingly the combination of agents according to the invention is particularly useful for treating microbial infections.

1. A method of administering chemotherapy comprising:
administering a first agent that attenuates Topoisomerase H (Topo II) activity; and
administering a second agent that inhibits Heat Shock Protein 90 (HSP90) activity,
wherein the first agent and the second agent are administered either contemporaneously or sequentially, and the first agent is selected from the group consisting of a Podophyllotoxin and derivatives and analogues thereof; an Anthracenedione and derivatives and analogues thereof, m-AMSA (amsacrine) and derivatives and analogues thereof, a Bisdioxopiperazine and derivatives and analogues thereof, a thiobarbiturate, Genistein and derivatives or analogues thereof, and Pyrazoloacridine and derivatives or analogues thereof.

2. The method of claim 1 wherein the first agent is a compound selected from the group consisting of:
(i) compounds that bind to Topo II and inhibit its activity, including competitive inhibitors and allosteric inhibitors,
(ii) compounds which prevent the transcription, translation or expression of Topo II, including ribozymes and antisense DNA molecules,
(iii) compounds which inhibit release of Topo II from intracellular stores, and
(iv) compounds which increase the rate of degradation of Topo II.

3. The method of claim 1 wherein the first agent is selected from the group consisting of etoposide (VP16) and teniposide.

4. The method of claim 1 wherein the first agent is Anthracenedione Mitoxantrone.

5. The method of claim 1 wherein the first agent is selected from the group consisting of ICRF-154, 159, 187 or 193.

6. The method of claim 1 wherein the first agent is the thiobarbiturate Merbarone or a derivative or analogue thereof.

7. The method of claim 1 wherein the second agent is a compound selected from the group consisting of:
(i) compounds that bind to Hsp90 and inhibit its activity, including competitive inhibitors and allosteric inhibitors,
(ii) compounds which prevent the transcription, translation or expression of Hsp90, including ribozymes and anti sense DNA molecules,
(iii) compounds which inhibit release of Hsp90 from intracellular stores, and (iv) compounds which increase the rate of degradation of Hsp90.

8. The method of claim 7 wherein the second agent is Geldanamycin or a derivative or analogue thereof.

9. The method of claim 8 wherein the second agent is 17-Allylamino, 17-demethoxygeldanamycin (17AAG).

10. The method of claim 7 wherein the second agent is Radicicol or a derivative or analogue thereof.

11. The method of claim 1 wherein the chemotherapy is for cancer treatment.

12. The method of claim 11 wherein the chemotherapy is for the treatment of solid tumours.

13. The method of claim 12 wherein the chemotherapy is for the treatment of bowel cancer, small cell and non-small cell lung cancer, head and neck cancer, breast cancer, bladder cancer or malignant melanoma.

14. The method of claim 11 wherein the chemotherapy is for the treatment of paediatric tumours.

15. The method of claim 14 wherein the chemotherapy is for the treatment of neuroblastoma, leukaemias and lymphomas.

16. The method of claim 11 wherein the first agent is etoposide and the chemotherapy is used in the treatment of cancers selected from the group consisting of: Adult Acute Myeloid Leukemia, Adult Hodgkin's Disease, Adult Non-Hodgkin's Lymphoma, AIDS-Related Lymphoma, Carcinoma of Unknown Primary, Childhood Acute Myeloid Leukemia, Childhood Brain Tumor, Childhood Cerebral Astrocytoma, Childhood Ependymoma, Childhood Hodgkin's Disease, Childhood Liver Cancer, Childhood Medulloblastoma, Childhood Non-Hodgkin's Lymphoma, Childhood Rhabdomyosarcoma, Childhood Supratentorial Primitive Neuroectodermal and Pineal Tumors, Childhood Visual Pathway and Hypothalamic Glioma, Endometrial Cancer, Ewing's Family of Tumors Including Primitive Neuroectodermal Tumor (PNET), Extragonadal Germ Cell Tumors, Gastric Cancer, Gastrointestinal Carcinoid Tumor, Gestational Trophoblastic Tumor, Kaposi's Sarcoma, Malignant Thymoma, Neuroblastoma, Non-small Cell Lung Cancer, Osteosarcoma/Malignant Fibrous Histiocytoma of Bone, Ovarian Epithelial Cancer, Ovarian Germ Cell Tumor, Pediatric Extracranial Germ Cell Tumor, Prostate Cancer, Retinoblastoma, Small Cell Lung Cancer, Testicular Cancer, Unusual Cancers of Childhood, and Wilms' Tumor and Other Childhood Kidney Tumors.

17. The method of claim 1 wherein the chemotherapy is for: antibacterial treatments, antifungal treatments, the treatment of AIDS/HIV, the treatment of multiple sclerosis, or the killing and inhibition of proliferation of any organism.

18. The method of claim 1 wherein the chemotherapy is for prophylactic treatment.

19. A delivery system for use in a gene therapy technique, said delivery system comprising:
(i) a first DNA molecule encoding for a protein which directly or indirectly attenuates Topoisomerase II activity; and
(ii) a second DNA molecule encoding for a protein which directly or indirectly inhibits Heat Shock Protein 90 activity;
wherein said DNA molecules are capable of being transcribed to allow the expression of said proteins and thereby be effective for chemotherapy.

20. The delivery system according to claim 19 configured for use in chemotherapy.

21. The delivery system of claim 20 wherein the chemotherapy is for cancer treatment; the treatment of solid tumors; the treatment of bowel cancer, small cell and non-small cell lung cancer, head and neck cancer, breast cancer, bladder cancer or malignant melanoma; the treatment of paediatric tumours; the treatment of neuroblastoma, leukaemias and lymphomas; the treatment of Adult Acute Myeloid Leukemia, Adult Hodgkin's Disease, Adult Non-Hodgkin's Lymphoma, AIDS-Related Lymphoma, Carcinoma of Unknown Primary, Childhood Acute Myeloid Leukemia, Childhood Brain Tumor, Childhood Cerebral Astrocytoma, Childhood Ependymoma, Childhood Hodgkin's Disease, Childhood Liver Cancer, Childhood Medulloblastoma, Childhood Non-Hodgkin's Lymphoma, Childhood Rhabdomyosarcoma, Childhood Supratentorial Primitive Neuroectodermal and Pineal Tumors, Childhood Visual Pathway and Hypothalamic Glioma, Endometrial Cancer, Ewing's Family of Tumors Including Primitive Neuroectodermnal Tumor (PNET), Extragonadal Germ Cell Tumors, Gastric Cancer, Gastrointestinal Carcinoid Tumor, Gestational Trophoblastic Tumor, Kaposi's Sarcoma, Malignant Thymoma, Neuroblastoma, Non-small Cell Lung Cancer, Osteosarcoma/Malignant Fibrous Histiocytoma of Bone, Ovarian Epithelial Cancer, Ovarian Germ Cell Tumor, Pediatric Extracranial Germ Cell Tumor, Prostate Cancer, Retinoblastoma, Small Cell Lung Cancer, Testicular Cancer, Unusual Cancers of Childhood, and Wilms' Tumor and Other Childhood Kidney Tumors; antibacterial treatments; antifungal treatments; the treatment of AIDS/HIV; the treatment of multiple sclerosis; the killing and inhibition of proliferation of any organism; and for prophylactic treatment.

22. A method of screening a first and a second compound, to test whether or not said compounds has efficacy for use in combination as a chemotherapy, comprising:
(a) exposing said compounds to Topoisomerase II and evaluating whether or not said compounds bind thereto;
(b) exposing said compounds to Heatshock Protein 90 and evaluating whether or not said compounds bind thereto; and
(c) selecting a first and second compound, wherein at least one compound binds to Topoisomerase II and at least one compound binds to Heatshock Protein 90 for use in combination as a chemotherapy.

23. A method of screening compounds, to test whether or not said compounds have efficacy for use in chemotherapy, comprising:
(a) exposing said compounds to Topoisomerase II and evaluating whether or not said compounds bind thereto;
(b) exposing said compounds to Heatshock Protein 90 and evaluating whether or not said compounds bind thereto; and
(c) selecting compounds that bind to Topoisomerase II and to Heatshock Protein 90 for use in chemotherapy.

24. The method according to claim 22 wherein the compound is screened using Topoisomerase II and Heatshock Protein 90 as binding partners in an interaction trap and evaluating whether or not said compound modulates binding.

25. The method according to claim 24 wherein the interaction trap is a yeast two-hybrid interaction trap.

26. The method according to claim 25 wherein yeast used in the interaction trap are permeable to the tested compounds.

27. A method of screening a compound, to test whether or not said compound is carcinogenic, comprising exposing said compound to Topoisomerase II and Heatshock Protein 90 to evaluate whether or not said compound promotes interaction between Topoisomerase II and Heatshock Protein 90.

28. An in vitro method for diagnosing whether or not a subject has, or is likely to develop cancer, comprising:
(i) detecting the level of activity or expression levels of HSP90 and Topoisomerase II from a sample of cells from said subject; and
(ii) comparing the level of activity or expression levels of HSP90 and Topoisomerase II in said sample relative to activity expression levels of HSP90 and Topoisomerase II from a non-cancerous sample.

29. An in vitro method for evaluating the suitability of chemotherapeutic treatment for administration to a subject, comprising:
(i) detecting the level of activity or expression levels of HSP90 and Topoisomerase 11 from a sample of cells from said subject; and
(ii) comparing the level of activity or expression levels of HSP90 and Topoisomerase II in said sample relative to activity expression levels of HSP90 and Topoisomerase II from a non-cancerous sample.

30. An in vitro method for monitoring the effectiveness of a chemotherapy for treating a subject, comprising:
(i) detecting the level of activity or expression levels of HSP90 and Topoisomerase II from a sample of cells from said subject; and
(ii) comparing the level of activity or expression levels of HSP90 and Topoisomerase II in said sample relative to activity expression levels of HSP90 and Topoisomerase II from a non-cancerous sample.

31. The method according to claim 23 wherein the compound is screened using Topoisomerase II and Heatshock Protein 90 as binding partners in an interaction trap and evaluating whether or not said compound modulates binding.

32. The method according to claim 31 wherein the interaction trap is a yeast two-hybrid interaction trap.

33. The method according to claim 32 wherein yeast used in the interaction trap are permeable to the tested compounds.
